(19) United States
(12) Patent Application Publication
(10) Pub. No.: US 2016/0061849 A1
(43) Pub. Date: Mar. 3, 2016

(54) LIPIDOMIC BIOMARKERS

(71) Applicants: UNITHER VIROLOGY, LLC, Silver Spring, MD (US); THE CHANCELLOR, MATERS AND SCHOLARS OF THE UNIVERSITY OF OXFORD, Oxford (GB)

(72) Inventors: Peter LAING, Cambridgeshire (GB); Raymond A. DWEK, Oxfordshire (GB); Stephanie POLLOCK, Oxfordshire (GB); Nicole ZITZMANN, Oxfordshire (GB)

(73) Assignees: UNITHER VIROLOGY, LLC, Silver Spring, MD (US); THE CHANCELLOR, MATERS AND SCHOLARS OF THE UNIVERSITY OF OXFORD, Oxford (GB)

(21) Appl. No.: 14/888,132

(22) PCT Filed: Apr. 30, 2014
(86) PCT No.: PCT/US14/36102

§ 371 Date: Oct. 30, 2015

(51) Int. Cl. G01N 33/92

(52) U.S. Cl. CPC G01N 33/92; G01N 2333/08; G01N 2800/26; G01N 2800/52

(57) ABSTRACT

Lipidomic markers for Hepatitis C and related conditions, treat hepatic fibrosis and hepatocellular carcinoma. An agent administered to such subject may be an cellular total fatty-acid content under iminosugar, which may be effective against hepatitis C. Such iminosugar may be, for example, one of N-substituted deoxynojrimycins and pharmaceutically acceptable salts thereof, N-substituted deoxygalactonojirimycins and pharmaceutically acceptable salts thereof and N-substituted Me-deoxygalactonojirimycins and pharmaceutically acceptable salts thereof. A method of assessing a Hepatitis C infection or a condition caused by or associated with said infection. This method comprises: obtaining a biological sample from a subject in need thereof; determining a level of at least one Hepatitis C lipidomic biomarker in said biological sample; and comparing said level of with a control level of said Hepatitis C lipidomic biomarker to assess the Hepatitis C infection or the condition caused by or associated with said infection in the subject.

### CROSS-REFERENCE TO RELATED APPLICATIONS

This application claims the benefit of U.S. Provisional Application No. 61/818,621, filed on May 2, 2013, the contents of which are hereby incorporated by reference in its entirety into the present disclosure.

### FIELD

The present application relates to diagnosing and/or prognosticating of diseases and medical conditions and in particular, to such diagnosing and/or prognosticating using lipidomic biomarkers.

### SUMMARY

One embodiment is a method of assessing a Hepatitis C infection or a condition caused by or associated with said infection. This method comprises: (a) obtaining a biological sample from a subject in need thereof; (b) determining a level of at least one Hepatitis C lipidomic biomarker in said biological sample; and (c) comparing said level of (b) with a control level of said Hepatitis C lipidomic biomarker to assess the Hepatitis C infection or the condition caused by or associated with said infection in the subject.

Another embodiment is a method for assessing a response to a therapy, comprising: (a) administering an agent to a subject in need thereof; (b) then obtaining a biological sample from the subject; (c) determining a desaturation index of at least one of glucosylceramide, lactosylceramide and sphingomyelin of the biological sample; and (d) comparing a value of the desaturation index to a control desaturation index value to assess a response to said agent, wherein a higher value of the determined desaturation index value compared to a control value indicates that the subject responds to the agent and/or that a therapeutic benefit is provided.

Yet another embodiment is a method of identifying of a Hepatitis C patient, who is unlikely to respond to a hepatitis C treatment comprising at least one of interferon and ribavirin. The method comprises: (a) obtaining a biological sample from a subject having a Hepatitis C infection; (b) determining a value of a desaturation index of at least one of glucosylceramide, lactosylceramide and sphingomyelin in lipoproteins of the biological sample; and (c) comparing the determined value to a control desaturation index value, wherein if the determined value is higher than the control value of the desaturation, the subject is likely not to respond to a hepatitis C treatment comprising at least one of interferon and ribavirin and/or is likely not to receive a therapeutic benefit.

### BRIEF DESCRIPTION OF THE DRAWINGS

FIG. 1 schematically illustrates selected iminosugars used in selected experiments: a)N-butyl-deoxynojirimycin (NB-DNJ; UV-1 or miglustat); b)N-(9-methoxynonyl)-deoxynojirimycin (N9-DNJ or UV-4); c)N-(5-adamantane-1-yl-methoxy-pentyl)-deoxynojirimycin (Adamantane-pentyl-dNM; AMP-DNM or AMP-DNJ); d)N—(N-butyl) deoxygalactojirimycin (NB-DGJ); e)N-(7-oxa-nonyl)-1,5,6-trideoxy-1,5-imino-D-galactitol (N-7-oxa-nonyl MeDGJ) (UT231-B); f)N—(N-{4′-azido-2′-nitrophenyl}-6-aminohexyl)deoxynojirimycin (NAP-DNJ or UV-5). The compounds share a common feature of having an endocyclic nitrogen atom in place of the oxygen of the corresponding sugar molecule. UV-1/NB-DNJ/miglustat is the active pharmaceutical ingredient (API) element of Zavesca®, which is an inhibitor of glucosylceramide used for the treatment of Gaucher disease. NB-DGJ, though having a galactose-type headgroup is a specific inhibitor of glucosylceramide synthase, which shows no inhibitory activity towards ER alpha glucosidases (unlike its epimeric analogue NB-DNJ which also inhibits glucosidase).

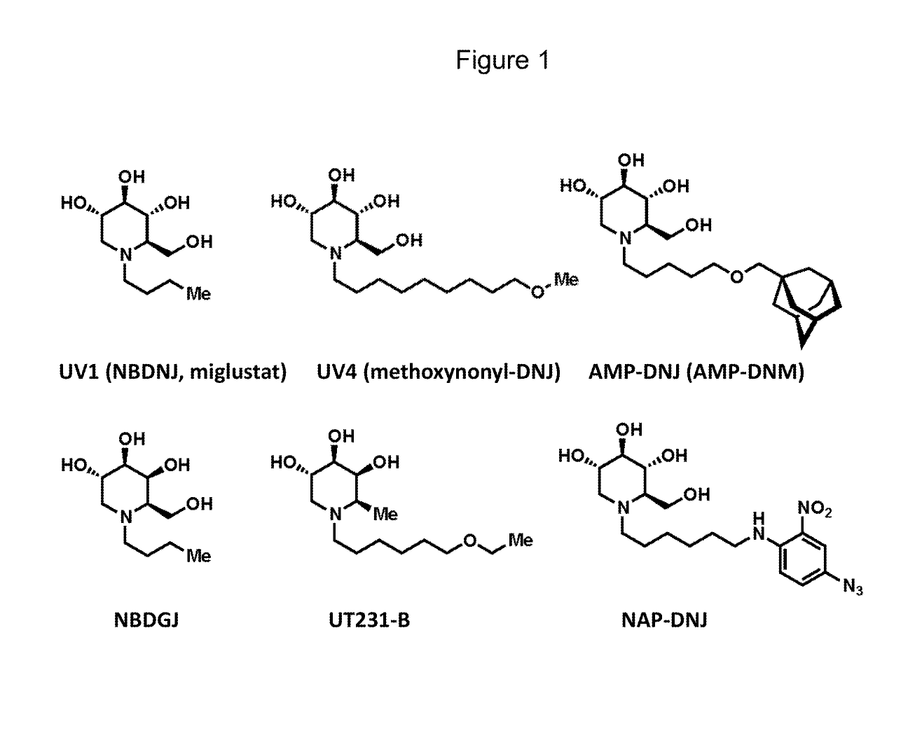

FIG. 2 provides measured total fatty acid content of hepatoma cells in the infected and uninfected states. The sum of fatty acid methyl esters for each test group after hydrolysis of cellular lipids is shown.

FIG. 3a-b provide results of analysis of total cellular fatty acid composition of hepatoma cells under treatment with various compounds from FIG. 1 and under influence of infection with hepatitis C virus (HCV). Numbers presented are percent composition for each fatty acid determined by analysis of fatty acid methyl esters. Data bars use the default settings of Microsoft Excel 2007 and are encoded ‘vertically’ to highlight changes per molecular species (a) in red and changes in the overall composition (b) in blue. (The former coding emphasizes changes in minor species that would otherwise be inconspicuous).

FIG. 4a-f provide comparisons of a percentage of a particular fatty acids in total cellular fatty acid composition between different types of cells. a) mead acid; b) Docosahexaenoic acid (DHA); c) oleic acid; d) linoleic acid; e) palmitoleic acid w9; f) palmitoleic acid w7. FIG. 4a-f demonstrates iminosugars influence fatty-acid composition in the uninfected state. Particular fatty acids from the fatty-acid methyl ester analysis are shown as percent composition—i.e. those which change either as a result of infection or iminosugar treatment. The infected samples are on the left of each panel (the left most seven bars).

FIG. 5a-b provide global desaturation index and global elongation index under the influence of infection and iminosugar compounds. Data from the global fatty acid analysis by fatty-acid methyl ester analysis (FIG. 3) are represented as desaturation and elongation indices of non-essential fatty acids as shown. Uninfected cells=light bars, infected cells=dark bars.

FIG. 6 presents measured cellular fatty acid composition of cholesterol esters for uninfected (left columns) and infected (right columns) cells. The most abundant species of cholesterol esters were measured as described in materials and methods and represented as percent compositional abundance. Light bars=uninfected, dark bars=infected cells.

FIG. 7 provides a table with cellular triglyceride composition under the influence of infection and iminosugar compounds. The percent compositional abundance of the various triglyceride species detected is tabulated with ‘horizontal’ data bars, depicting changes in overall abundance of each molecular species.

FIG. 8 presents changes in triglyceride composition in the infected state. Changes in triglyceride molecular composition are represented as ‘fold-change’ from the uninfected state, which highlights rare species that are markedly changed in abundance. Dark (blue) bars=decreased by infection, light (red) bars=increased by infection.

FIG. 9 provides a table with molecular composition of phosphatidylcholine (ester form) under the influence of infection and iminosugars. Changes in abundance of PC molecular species are tabulated—arrows indicate increase or decrease in the infected state.

FIG. 10 presents plots of phosphatidylcholine (ether form) fatty acid composition under the influence of infection and iminosugars. The compositional abundance of ether forms of PC (plasmalogen forms) is depicted.

FIG. 11a-g present plots of fatty acid composition of lysophosphatidylcholine under the influence of infection and iminosugars. The compositional abundance (percent) of each lyso-PC species is indicated. Light bars=uninfected, dark bars=infected cells.

FIG. 12 provides a table of phosphatidylethanolamine (PE) molecular composition under the influence of infection and iminosugars. The percent abundance of PE species is tabulated with data bars encoded vertically to emphasize changes between the infected and uninfected state. Arrows indicate species that are increased or decreased by infection.

FIG. 13 provides a table with phosphatidylserine (PS) molecular species under the influence of infection. In the case of PS there were no significant changes in the infected or uninfected state under the influence of iminosugars. The percent abundance of molecular species of PS, and changes therein between the uninfected and infected state are shown as ratios.

FIG. 14 provides a table of phosphatidylinositol (PI) molecular species under the influence of infection and iminosugars. PI has only four molecular species. Their abundance is vertically encoded with data-bars in order to illustrate the change in abundance of the individual molecular species upon infection.

FIG. 15a-f present plots of cellular abundance of sphingolipids under the influence of infection and iminosugars. The cellular abundance of the sphingolipids ‘ceramide’ (Cer), glycosylceramide (GlcCer) and lactosylceramide (LacCer) is indicated in nanomoles per mg of protein, under the various treatments.

FIG. 16 provides results of principal component and discriminant analysis of glucosylceramide. Principal component analysis (left) and discriminant analysis were applied to the entire dataset of treated versus untreated, and iminosugar treated versus untreated cells. PCA identified that the difference between GlcCer 24:1 and GlcCer 24:0 could explain most of the variation in the dataset. Untreated samples from infected or uninfected cultures were not distinguished in this analysis, however, all iminosugar treatments (in the uninfected or infected state) were distinguishable from untreated samples (ellipses represent 95% confidence intervals).

FIG. 17 provides results of principal component (PCA) and discriminant analysis of phosphatidylcholine molecular species under the influence of infection and iminosugars. PCA and discriminant analysis distinguished clearly PC molecular species, forming two major groups ‘infected’ (left half of the square) and uninfected (right half of the square), distinguished from one another in the F1 dimension (accounting for 91.3% of the variation in the dataset), which in this case represents a decrease in monounsaturated species (e.g. PC32:1), while saturated PC32:0 and 34:0 and PUFA-enriched PCs (PC34:4 and PC38:5) were enriched by infection (refer also to FIG. 9 arrowed columns). Unlike changes observed by PCA and discriminant analysis for GlcCer, in the case of PC, infected iminosugar-treated cells could not be distinguished from untreated infected cells (such differences accounting for only a fraction of the F2 (vertical) dimension which comprised only 6.79% of the variation in the dataset. Similar results were obtained with PE, i.e. iminosugar-treated infected cells were not clearly distinguished from untreated infected cells—the effects of infection predominating over the effects of iminosugar treatment.

FIG. 18a-d presents plots of desaturation index (24:1/24:0) for GlcCer in cells under the influence of infection and iminosugar compounds. Following PCA and discriminant analysis described in FIG. 16, which identified variation between 24:1 and 24:0 species (at delta-9) as the major variant in the dataset, the corresponding GlcCer desaturation index was plotted for each of the cell samples and treatments (a, b). Note that the desaturation index for GlcCer, though it is ‘delta-9’, does not change between the infected and the uninfected state (reflecting the findings of PCA and discriminant analysis). Desaturation index for LacCer (which is produced from GlcCer) is also plotted—(c, d).

### DETAILED DESCRIPTION

Unless otherwise specified “a” or “an” means one or more.

The present inventors discovered that one may assess a Hepatitis C infection and/or an associated condition, such as liver fibrosis, cirrhosis, and hepatocellular carcinoma, by determining a level of one or more lipidomic biomarkers, which may be a lipid metabolite, in a biological sample obtained from a subject and comparing the determined level with a control level.

The term “lipidomic marker” or “lipidomic biomarker” may refer to a particular difference in a lipid composition between a biological sample from a subject with a disease or condition, such as hepatitis C and/or an associated condition, and a control biological sample, which may be a sample of one or more healthy individuals or a sample of one or more individuals without the disease or condition. In some embodiments, the term “lipidomic marker” or “lipidomic biomarker” may refer to a particular difference in absolute abundance of one or more lipid components or metabolites thereof between a biological sample from a subject with a disease or condition, such as hepatitis C and/or an associated condition, and a control biological sample. Yet in some embodiments, the term “lipidomic marker” or “lipidomic biomarker” may refer to a particular difference in relative abundance between lipid components or metabolites thereof or between a biological sample from a subject with a disease or condition, such as hepatitis C and/or an associated condition, and a control biological sample.

“Biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived there from and the progeny thereof. Additionally, the term may encompass circulating tumor or other cells. The term specifically encompasses a clinical sample, and further includes cells in cell culture, cell supernatants, cell lysates, serum, plasma, urine, amniotic fluid, biological fluids, and tissue samples. The term also encompasses samples that have been manipulated in any way after procurement, such as treatment with reagents, solubilization, or enrichment for certain components

The biological sample may be a sample of a body fluid or a body tissue of the subject. For example, the biological sample may be a sample of blood, plasma, serum, saliva, bile, urine, feces or cerebrospinal fluid or samples derived from cells, tissues, or organs, such as a liver, from the subject. In many embodiments, it may be preferred to use blood, plasma or serum as a biological sample. A variety of techniques are available for obtaining a biological sample.

“Individual,” “subject,” “host,” and “patient,” used interchangeably herein, refer to any animal subject, such as a mammalian subject for whom diagnosis, treatment, or therapy is desired. In one preferred embodiment, the individual, subject, host, or patient is a human. Other subjects may include, but are not limited to, cattle, horses, dogs, cats, guinea pigs, rabbits, rats, primates, woodchucks, ducks, and mice.

In some embodiments, the biological sample may be pretreated prior to determining the level of the lipidomic biomarker. Such pretreatment may, for example, involve separating at least one fraction of the biological sample and performing determination of the level of the lipidomic marker in the separated fraction. Such a separation fraction may be, for example, a lipoprotein fraction, such as a very low density lipoprotein fraction or a low-density protein fraction, a glyceride fraction, such as a triglyceride fraction, or a phospholipid fraction. In some embodiments, the separation fraction may be a high density lipoprotein fraction or exosome fraction, see e.g. Keller, Sanderson et al (see REFERENCES section below). For separation of a particular fraction, a suitable separation technique, such as centrifugation, extraction, fractioning, ultrafiltration, protein precipitation, or chromatographical separation, may be used.

Yet in some embodiments, the determining the level of the lipidomic marker may be performed on an unpretreated or unfractionated sample.

In some embodiments, it may be preferred to perform determining the level of the lipidomic marker an unpretreated or unfractionated sample obtained from a subject in a fasted state, which may mean at least 1 hour or at least 1.5 hours or at least 2 hours or at least 2.5 hours or at least 3 hours after the latest meal, for example, in the morning before breakfast.

Yet in some embodiments, determining the level of the lipidomic marker an unpretreated or unfractionated sample obtained from a subject in a fasted state obtained from a subject in a postprandial state.

Determining the level of a lipidomic biomarker may be quantitative or semi-quantitative. In some embodiments, quantitative determination may involve determining an absolute amount or concentration of one or more lipid metabolites. Yet in some embodiments, quantitative determination may involve determining a relative amount or concentration of one or more lipid metabolite's with respect to one or more other metabolites. For example, in some embodiments, one may determine a ratio of the amount or concentration of at least one metabolite A with respect to the amount or concentration of at least one metabolite B.

Determining the level of a lipidomic marker may be performed using a number of techniques. In some embodiments, determining the level of a lipidomic marker may involve using a chromatographic technique, such as liquid chromatography (LC), high performance liquid chromatography (HPLC), gas chromatography (GC), thin layer chromatography, size exclusion or affinity chromatography. In some embodiments, determining the level of a lipidomic marker may involve using a mass spectrometry technique, such as gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Suitable techniques are disclosed in, e.g., Nissen, Journal of Chromatography A, 703, 1995: 37-57, U.S. Pat. No. 4,540,884 or U.S. Pat. No. 5,397,894.

In some embodiments, determining the level of a lipidomic marker may involve using one of the following techniques: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionization detection (FID). In some embodiments, one may use fatty acyl ester analysis for determining, for example, fatty acyl composition of a particular lipoprotein fraction, such as a particular blood lipoprotein fraction. In some embodiments, LC-MS may be used for determining individual lipid species, such as phospholipids or sphingolipids.

In some embodiments, determining the level of a lipidomic marker may involve using gas chromatography with online mass spectrometry (GCMS) and/or LCMS2 (high performance liquid chromatography with online two-dimensional mass spectrometry) with suitable internal standards using software tools, such as lipid mass spectrum analysis software (LIMSA), see e.g. Haimi et al. Methods Mol. Biol. 2009, 580, 285-94, for data processing.

In some embodiments, determining the level of a lipidomic marker may involve a specific chemical or biological essay. The essay may utilize one or more agents that can specifically recognize the chemical structure of a lipid metabolite or are capable of specifically identifying the lipid metabolite based on its capability to react with other compounds or its capability to elicit a response in a biological read out system. For example, in some embodiments, an immunoassay may be used wherein an agent, such as an antibody, that is specific for the analyte in question is used to measure the abundance of the target species.

In some embodiments, determining the level of a lipidomic marker may involve using two or more techniques disclosed above.

In some embodiments, a Hepatitis C lipidomic marker may be an abundance, i.e. an amount or concentration, of Mead acid in the biological sample. A higher value of the Mead acid's abundance compared to a control abundance value may indicate that the subject has a Hepatitis C infection or a condition associated with or caused by such infection.

In some embodiments, the Mead acid's abundance may be used as a biomarker of Hepatocellular Carcinoma. In such a case, a higher value of the Mead acid's abundance compared to a control abundance value may indicate that the subject has Hepatocellular Carcinoma.

The sample for determining Mead acid's abundance may be a sample of a biological fluid, such as plasma, blood or serum. In some embodiments, determining Mead acid's abundance may be performed on an untreated or unfractionated sample. Yet in some embodiments, determining Mead acid's abundance may be performed on a particular fraction of the sample, such as, for example, a very low density lipoprotein fraction.

In some embodiments, a biological sample for determining Mead acid's abundance may be obtained when the subject is in a fasted state, which may mean at least 1 hour or at least 1.5 hours or at least 2 hours or at least 2.5 hours or at least 3 hours after the latest meal. In some embodiments, it may be preferred that the fasting time does not exceed 24 hours. Yet in some embodiments, a biological sample for determining Mead acid's abundance may be obtained when the subject is in a postprandial state.

In some embodiments, an abundance, i.e. an amount or concentration, of at least one non-essential fatty acid by-product of de novo lipogenesis, such as palmitoleic acid (C16:1 omega 9 and omega 7) and oleic acid (C18:1 omega 9) may be used as a biomarker of Hepatitis C or a condition associated with or caused by such infection. In such a case, a lower value of the determined abundance compared to a control abundance value may indicate that that the subject has a Hepatitis C infection or a condition associated with or caused by such infection.

In some embodiments, a desaturation index of non-essential fatty acids, which may be, for example, a desaturation index of non-essential fatty acids present in lipids of blood lipoprotein fraction(s), such as VLDL fraction, may be used as a biomarker of Hepatitis C or a condition associated with or caused by such infection. In such a case, a lower value of the desaturation index compared to a control desaturation index value may indicate the subject has a Hepatitis C infection or a condition associated with or caused by such infection. Such a biomarker may be a better measure of liver damage compared to some other biomarkers, such as viraemia. The desaturation index may be, for example, a ((16:1 ω-7+16:1ω-9)/16:0) ratio, i.e. a ratio between a combined abundance of 16:1 ω-7 and 16:1ω-9 fatty acids with to an abundance of 16:0 fatty acid.

In some embodiments, a degree of elongation of non-essential fatty acids in the biological sample may serve as serve as a biomarker of a Hepatitis C infection or a condition associated with or caused by such infection. In such a case, a higher value of the elongation degree determined for the biological sample compared to a control elongation value may indicate the subject has a Hepatitis C infection or a condition associated with or caused by such infection. Such biomarker may be a better indicator of liver damage compared to some other biomarkers, such as viraemia. The elongation degree may be determined, for example, using a (18:1 omega-7/16:1 omega-7) ratio, i.e. a ratio between an abundance of 18:1 omega-7 fatty acid and 16:1 omega-7 fatty acid.

In some embodiments, a lipidomic biomarker of a Hepatitis C infection or a condition associated with or caused by such infection may be an abundance of at least one polyunsaturated omega-6 and omega-3 fatty acid, such as arachidonic acid and docohexaenoic acid. In such a case, a higher value of such abundance determined in the biological sample compared with a control abundance value, which may be an abundance value for one or more healthy individual not infected with HCV, may indicate that the subject has a Hepatitis C infection or a condition associated with or caused by such infection. Such biomarker may be a better indicator of progress of liver damage compared to some other biomarkers, such as viraemia.

In some embodiments, an abundance, i.e. a concentration or amount, of one or more fatty acids in a cholesterol ester profile of the biological sample may serve as a lipidomic biomarker of a Hepatitis C infection or a condition associated with or caused by such infection. For measuring a cholesterol ester profile, cholesterol esters may be purified from the biological sample using a separation technique, such as chromatographic purification. In certain cases, such fatty acid may be at least one polyunsaturated essential omega-3 or omega-6 fatty acid, such as a 20:4 fatty acid, a 20:5 fatty acid, a 22:6 fatty acid and a 22:5 fatty acid. In such a case, a higher value of the abundance determined in the one or more of such polyunsaturated fatty acids compared to a control abundance value may indicate that the subject has a Hepatitis C infection or a condition associated with or caused by such infection. Such biomarker may be a better indicator of the progress of liver damage compared to some other biomarkers, such as viraemia. In certain cases, the paucity of certain fatty acids in the cholesterol ester profile may be indicative of the presence or effect of HCV upon liver cells in an infected individual. In such cases, the fatty acid may be at least one monounsaturated fatty acid, which may be, for example, a 16:1 fatty acid and a 18:1 fatty acid. In such a case, a lower value of the abundance determined in the one or more of such monounsaturated fatty acids compared to a control abundance value may indicate that the subject has a Hepatitis C infection or a condition associated with or caused by such infection.

In some embodiments, an abundance, i.e. a concentration or an amount, of one or more triglycerides in the biological sample may serve as a lipidomic biomarker of a Hepatitis C infection or a condition associated with or caused by such infection. Such triglyceride may be, for example, C54:5-C18:0 triglyceride; C54:6-C18:1 triglyceride; C56:5-C20:4 triglyceride or C56:7-C22:6 triglyceride. In such a case, a higher value of the abundance of the triglyceride compared to a control abundance value may indicate that the subject has a Hepatitis C infection or a condition associated with or caused by such infection.

In some embodiments, determining the abundance of a triglyceride biomarker may be performed in an untreated or unfractionated biological sample, such as a plasma, blood or serum sample. Yet in some embodiments, determining the abundance of a triglyceride biomarker may be performed in a fraction of the biological sample, such as a very low density lipoprotein fraction and a triglyceride fraction. In some embodiments, determining the abundance of a triglyceride biomarker may be performed in a biological sample obtained from a subject in a fasted state, i.e. at least 1 hour or at least 1.5 hours or at least 2 hours or at least 2.5 hours or at least 3 hours after the latest meal. Yet in some embodiments, determining the abundance of a triglyceride biomarker may be performed in a biological sample obtained from a subject in a postprandial state. For determining an abundance of C54:5-C18:0 triglyceride; C56:5-C20:4 triglyceride or C56:7-C22:6 triglyceride, it may be preferred to use an unfractionated biological sample, such as plasma, blood or serum sample, obtained from a subject in a fasted state. Alternatively, for these biomarkers, one may use a fraction of a biological sample, such as a very low density lipoprotein fraction and a triglyceride fraction. Determining an abundance of C54:6-C18:1 triglyceride may be performed in a unfractionated biological sample, such as plasma, blood or serum sample, obtained from a subject in a fasted or postprandial state.

In some embodiments, an abundance, i.e. a concentration or an amount, of one or more fatty acids among phospholipids of the biological sample may serve as a lipidomic marker of a Hepatitis C infection or a condition associated with or caused by such infection. In some embodiments, an abundance, i.e. a concentration or an amount, of one or more fatty acids among ester bonded phospholipids of the biological sample may serve as such a lipidomic marker. For example, in some embodiments, an abundance of at least one fatty acid in a diester form of phosphatidylcholines of the biological sample may be the lipidomic marker. Such fatty acid may be selected, for example, from a PC 32:1 species, a PC 32:0 species, a PC 34:0 species, a PC 34:4 species and a PC 34:5 species. In such a case a higher value of the abundance of at least one of the PC 32:0 species, the PC 34:0 species, the PC 34:4 species and the PC 34:5 species compared to a respective control value may indicate that the subject has a Hepatitis C infection or a condition associated with or caused by such infection. A lower value of the abundance of the 32:1 species compared to a respective control value may also indicate that the subject has a Hepatitis C infection or a condition associated with or caused by such infection.

In some embodiments, an abundance, such as a concentration or amount, of at least one fatty acid in a diester form of phosphatidylethanolamines of the biological sample may be used as a lipidomic marker of a Hepatitis C infection or a condition associated with or caused by such infection. Such fatty acid may be selected, for example, from a) a Mead acid; b) at least one palmitoleic acids, such as 16:1 omega-7 and omega-9 acids; or c) at least one of essential omega-3 or omega-6 fatty acids, such as 20:3 omega-3, 20:4 omega-6, 20:5 omega-3, 22:6 omega-3, 22:5 omega-3 and 22:4 omega-6. In such a case, a higher value of the abundance determined of the Mead acid or the at least one essential omega-3 or omega-6 compared to its respective control abundance value may indicate that the subject has a Hepatitis C infection or a condition associated with or caused by such infection. A lower value of the abundance of the at least one palmitoleic acid compared to its respective control value may also indicate that the subject has a Hepatitis C infection or a condition associated with or caused by such infection.

In some embodiments, an abundance, i.e. a concentration or amount, of at least one fatty acid in a diester form of phosphatidylserines of the biological sample may serve as a lipidomic marker of a Hepatitis C infection or a condition associated with or caused by such infection. Such fatty acid may be, for example, a 38:3 species or a 40:6 species. In such a case, a higher value of the abundance of the at least one of the 38:3 species and the 40:6 species compared to its respective control value may indicate that the subject has a Hepatitis C infection or a condition associated with or caused by such infection.

In some embodiments, an abundance, i.e. a concentration or amount, of at least one fatty acid in a diester form of phosphatidylionositol of the biological sample may serve as a lipidomic marker of a Hepatitis C infection or a condition associated with or caused by such infection. Such fatty acid may be, for example, a PI 38:3 species; a PI 36:4 species, a PI 38:4 species or a PI 38:5 species. In such a case, a higher value of the determined abundance of the PI 38:3 species compared to a respective control abundance value or a lower value of the abundance of the at least one of the PI 36:4 species, the PI 38:4 species and the PI 38:5 species compared to a respective control abundance value may indicate that the subject has a Hepatitis C infection or a condition associated with or caused by such infection.

In some embodiments, an abundance, i.e. a concentration or amount, of at least one fatty acid among at least one of lyso-phosphatidylcholines of the biological sample may serve as a lipidomic marker of a Hepatitis C infection or a condition associated with or caused by such infection. Such fatty acid may be a 16:1 species, a 16:0 species, a 20:4 species or a 22:6 species. In such a case higher value of the determined abundance of at least one of the 16:0 species, the 20:4 species and the 22:6 species compared to its respective control value or a lower value of the 16:1 species compared to its respective control value may indicate that the subject has a Hepatitis C infection or a condition associated with or caused by such infection.

In some embodiments, for determining abundances of fatty acids among ester bonded phosphadylcholines and ester bonded phosphatylethanolamine, it may be preferred to use a fraction of the biological sample, such as a very low density lipoprotein fraction of the biological sample.

The present biomarkers may be used for diagnosing a Hepatitis C infection or a condition associated with or caused by such infection. In such a case, the control level or the control value may refer to a level or value of the biomarker determined in a healthy individual, who does not have the Hepatitis C infection or a condition associated with or caused by such infection. The control level or value may be also a level or value averaged over a pool of healthy individuals.

The present biomarkers may be used for assessing the progression or regression a Hepatitis C infection or a condition associated with or caused by such infection. In such a case, the control level or the control value may refer to a level or value of the biomarker determined in the same subject at an earlier time.

The present biomarkers may be used for assessing an effect of an agent on a Hepatitis C infection or a condition associated with or caused by such infection. In such a case, the control level or the control value may refer to a level or value of the biomarker determined, for example, prior to administering the agent to the subject.

The present biomarkers may be also used for assessing a response to a treatment for the Hepatitis C infection or a related condition. In such a case, the control level or the control value may refer to a level or value of the biomarker determined, for example, prior to administering the treatment to the subject.

The present inventors also discovered a response to a therapy, which may involve administering a therapeutic agent to a subject, using a lipidomic biomarker, which may be a desaturation index in at least one of glucosylceramide, lactosylceramide and sphingomyelins of a biological sample obtained from the subject. A higher value of the determined desaturation index value compared to a control value, i.e. a value determined in a sample of the subject obtained prior to administering the agent, may indicate that the subject responds to the agent. Such a desaturation index may be a 24:1/24:0 ratio.

In some embodiments, the desaturation index may be determined in a very low density lipoprotein fraction of the biological sample.

In some embodiments, the desaturation index may be determined in one of glucosylceramide and lactosylceramide. Yet in some embodiments, the desaturation index may be determined in both of glucosylceramide and lactosylceramide.

In some embodiments, a response to a therapy may be further assessed using an abundance, i.e. a concentration or amount, of glucosylceramide of the biological sample in addition to the desaturation index marker. In such a case, a reduced abundance of glucosylceramide, particularly in the VLDL blood fraction, compared to a control value, i.e. a value prior to administering the agent, may indicate that the subject responds to the therapy.

In some embodiments, the subject may be a subject with a Hepatitis C infection or an associated condition, such as a hepatic fibrosis or hepatocellular carcinoma. An agent administered to such subject may be an iminosugar, which may be effective against hepatitis C. Such iminosugar may be, for example, one of N-substituted deoxynojrimycins and pharmaceutically acceptable salts thereof, N-substituted deoxygalactonojirimycins and pharmaceutically acceptable salts thereof and N-substituted Me-deoxygalactonojirimycins and pharmaceutically acceptable salts thereof. Exemplary iminosugars may include, but not limited to, N-butyl deoxynojirimycin and a pharmaceutically acceptable salt thereof and N-(7-oxa-nonyl)-1,5,6-trideoxy-1,5-imino-D-galactitol and a pharmaceutically acceptable salt thereof. Iminosugars effective against hepatitis C are disclosed, for example, in U.S. Pat. Nos. 7,612,093 and 6,465,487.

In some embodiments, the subject may be a subject with a lysomal storage disorder, such as Gaucher disease or Niemann-Pick type-C disease. An agent administered to such subject may be an iminosugar, which may be effective against a lysosomal storage disorder. Such iminosugar may be, for example, one of N-substituted deoxynojrimycins and pharmaceutically acceptable salts thereof, N-substituted deoxygalactonojirimycins and pharmaceutically acceptable salts thereof and N-substituted Me-deoxygalactonojirimycins and pharmaceutically acceptable salts thereof. Exemplary iminosugars may include, but not limited to, N-butyl deoxynojirimycin and a pharmaceutically acceptable salt thereof; N-nonyl deoxynojirimycin and a pharmaceutically acceptable salt thereof and N-butyl deoxygalactonojirimycin and a pharmaceutically acceptable salt thereof. Iminosugars effective against hepatitis C are disclosed, for example, in U.S. Pat. Nos. 5,472,969; 5,525,616; 5,580,884; 5,656,641; 5,786,369; 5,798,366; 5,801,185; 6,291,657; 6,465,488; 6,495,570; 6,610,703; 6,660,749; 6,696,059; 7,348,000.

In some embodiments, the subject may be a subject with diabetes, e.g. with a type II diabetes. An agent administered to such subject may be an insulin sentisizing agent, which may be, for example, an iminosugar, a biguanide or a thiazolidinedione. One example of insulin sentisizing iminosugar may be N-(5-adamantane-1-yl-methoxypentyl)-DNJ and a pharmaceutically acceptable salt thereof. Examples of thiazolidinedione insulin sensitizing agents include, but not limited to, Pioglitazone and Rosiglitazone. One non-limiting example of a biguanide insulin-sensitizing agent is metformin. In general, biguanides and insulin sentisizing agents are generally known to those of ordinary skill in the art.

The present inventors also hypothesize that a Hepatitis C patient, who is unlikely to respond to a hepatitis C treatment comprising administering at least one of interferon, such as pegylated interferon alpha, and ribavirin, (a non-responder patient) may be identified through determining a value of a desaturation index of at least one of glucosylceramide, lactosylceramide and sphingomyelin in lipoproteins of a biological sample obtained from such patient. If the determined value of the desaturation index turns out to be higher than a control desaturation index value, the patient is likely not to respond to a hepatitis C treatment comprising at least one of interferon and ribavirin. In such a case, the identified non-responder patient may be administered an alternative therapy, in addition to the interferon and/or ribavirin therapy or instead of the interferon and/or ribavirin therapy. Such alternative therapy may involve administering an iminosugar, which may be effective against hepatitis C, such as those disclosed in U.S. Pat. Nos. 7,612,093 and 6,465,487. The desaturation index may be a 24:1/24:0 ratio. In some embodiments, the alternative therapy may include administering a direct acting antiviral agent, which may be, for example, an inhibitor of HCV protease, such as Telaprevir or Boceprevir, or a polymerase inhibitor.

In some embodiments, the desaturation index may be determined in a very low density lipoprotein fraction of the biological sample.

In some embodiments, the desaturation index may be determined in one of glucosylceramide and lactosylceramide. Yet in some embodiments, the desaturation index may be determined in both of glucosylceramide and lactosylceramide.

The present application also provides kits, which may include (a) one or more reagents for measuring a level of one or more lipidomic biomarker and (b) instructions for use. Such a kit may provide 1, 2, 3, 4, 5, 10, 15, 20, or more reagents for measuring the level of 1, 2, 3, 4, 5, 10, or more lipidomic biomarkers. In some embodiments, the kit may include one or more reagents for an immunoassay. In some embodiments, the kit may include one or more reagents for an MS assay. In some embodiments, the reagent may be an antibody to lipid metabolite, such as a fatty acid. Methods of making antibodies are known to those of ordinary skill in the art.

In some aspects, the kit may comprise (a) an antibody to a lipid metabolite, such as a fatty acid; and (b) instructions for use. In some embodiments, the kit may further comprise: (c) a second antibody to a second lipid metabolite, such as a fatty acid. In some embodiments, the kit further comprises (d) a third antibody to a third lipid metabolite, such as a fatty acid.

The present invention can be illustrated in more detail by the following example, however, it should be understood that the present invention is not limited thereto.

### Example

The lipid composition of hepatoma cells was studied under the influence of infection with replication-competent hepatitis-C virus (HCVcc) and under the influence of treatment with various antiviral iminosugar compounds which are inhibitors of ER glucosidases and/or of glucosylceramide synthase. In the absence of infection, untreated hepatoma cells showed markedly elevated levels of unsaturated non-essential fatty acids in the global fatty acid profile of the cells indicating a constitutive state of essential fatty acid deprivation. In particular, Mead acid (eicosatrienoic acid, 20:3 omega-9) was highly elevated. Infection markedly inhibited de novo biosynthesis of fatty acids (evident from decreased content of Mead acid and monounsaturated fatty acids) and brought about enrichment in highly polyunsaturated essential omega-3 and omega-6 fatty acids. Infection elevated the fatty acid content of cells and markedly changed the fatty-acyl composition of phospholipids, triglycerides and cholesterol esters, increasing length and saturation of endogenously synthesized non-essential fatty acyl chains, and increasing the incorporation of essential highly polyunsaturated fatty acids into membrane and storage lipid forms Iminosugar compounds reduced the abundance of glucosylceramide, but surprisingly also increased its unsaturation. These changes in abundance and saturation of glucosylceramide in response to iminosugars were present in both the infected and uninfected state. The iminosugars, in contrast to the effects of HCV, stimulated de novo lipogenesis and Mead acid production, but only in the uninfected state. These newly observed changes in cellular lipid composition, indicative of oncogenic transformation, HCV infection and response to iminosugar treatments, may serve as diagnostic and/or prognostic markers of hepatitis-C disease activity and for the diagnosis of hepatocellular carcinoma.

### Hepatitis C and Related Conditions

Approximately 3 percent of the world's population is infected with hepatitis-C virus (Marcellin 1999, for citations see section REFERENCES below) which is a leading cause of chronic liver disease including liver fibrosis, cirrhosis, and hepatocellular carcinoma. Moreover, hepatitis-C virus infection is the most common indication for liver transplantation in the US and Europe (Chen and Morgan 2006). The infection is ‘curable’ (i.e. there is a sustained virological response) in about 50% of cases by combination therapy with PEGylated interferon alpha in combination with Ribavirin (interferon+ribavirin), although the side effects of such therapy, which requires up to one year of treatment, are significant (e.g. the influenza-like symptoms of interferons) (Awad, Thorlund et al. 2010; Pawlotsky 2011). Cure rates are increasing with new ‘direct acting’ antiviral agents recently licensed (e.g. the protease inhibitors Telaprevir and Boceprevir) and with drugs in development such as Gilead's GS-7977 (formerly PI-7977), a nucleotide analog polymerase inhibitor. These new drugs hold considerable promise, but the extent to which they will be successful long-term in the face of the high mutability of the virus, and the extent to which they will be successful in combination with other drugs that might be needed to prevent resurgence of the infection while avoiding the use of interferons and their attendant side effects, remains to be seen (Pawlotsky 2011). Also, the newly licensed drugs are expensive, and insurance and healthcare budgets are limited—such that it may be important, for pharmacoeconomic reasons, to know which patients are likely to require the expensive new therapies, and who may fare adequately on the historically-established standard of care (interferon+ribavirin). Likewise, it may be important to be able to predict which patients will experience a rapid progression of disease and are likely, sooner than others, to require more-aggressive treatment, or liver transplantation.

One might expect that the amount of hepatitis-C virus in the bloodstream would be a good measure of the severity of underlying liver disease in infected patients. However, this has proven not to be the case: i.e. there is no clear relationship between viraemia (quantity of virus in the blood, assessed by the relatively non-invasive method of blood sampling), compared to liver biopsy which (though painful and posing significant risks to the patient) is regarded as the most reliable indicator of liver pathology (Hollingsworth R C 1996). To date, the focus of non-invasive prognostic investigations has been to detect the onset and progress of fibrosis (a predictor of subsequent cirrhosis) by measuring protein biomarkers in the blood. To this end, panels of protein biomarkers have been developed (e.g. the FibroTest, known as FibroSure in the USA) (Castéra, Vergniol et al. 2005) (Gangadharan, Bapat et al. 2012). These protein biomarker tests may have the ability to detect fibrosis indicating the onset of cirrhosis at the preclinical stage (FibroTest), and furthermore protein biomarkers such as the novel ones we have discovered have the potential to act as useful indicators of disease activity. Increasingly however, there is a need to obtain an early indication of the impact of the virus on liver pathology, using non-invasive or minimally-invasive methods. Also, there is an interest in identifying biomarkers or biomarker panels, that would predict responsiveness to a particular treatment, in order to ensure that patients are treated with appropriate drugs or drug combinations that they are most likely to respond to, i.e. so-called ‘personalized’ or ‘stratified’ medicine. Such endeavors balance the best interests of the patient, with the best interests of society (including other patients with different diseases with similar magnitude of medical need), given inherently limited healthcare budgets.

The best-established example to date of a biomarker that predicts treatment response in hepatitis-C virus infection is polymorphism of the IL28B gene which is predictive of sustained virological response to PEG-interferon-alpha in combination with Ribavirin (which until recently was the ‘Standard of Care’ for the treatment of hepatitis-C patients). However, the predictive value of IL28 polymorphism is not so strong, on its own, that it is yet used in the clinical judgment of deciding whether a patient will respond to any particular treatment regimen. It seems likely however, that IL28 polymorphism may have additive or synergistic value with other genetic polymorphisms or could be used likewise in combination with other biomarker strategies (e.g. protein based biomarkers) to help make decisions about treatment, in an emerging therapeutic environment where there is greater choice of drugs as a result of newly licensed drugs and new drugs in development (Clark and Muir 2012). To date however, biomarker strategies for hepatitis-C virus infection have concentrated upon protein and genetic markers, and have not, so far, investigated or identified the possibility of using lipid biomarkers.

Hepatitis-C virus is remarkably dependent upon cellular lipid metabolism, particularly cholesterol metabolism, of the hepatocyte for its replicative cycle (Barba, Harper et al. 1997; Sagan, Rouleau et al. 2006; Aizaki, Morikawa et al. 2008; Amemiya, Maekawa et al. 2008; Burlone and Budkowska 2009; Lyn, Kennedy et al. 2009; McLauchlan 2009; Ogawa, Hishiki et al. 2009; Diamond, Syder et al. 2010; Herker, Harris et al. 2010; Syed, Amako et al. 2010; Merz, Long et al. 2011; Miyoshi, Moriya et al. 2011; Clark, Thompson et al. 2012; Moriishi and Matsuura 2012; Rodgers, Villareal et al. 2012). In vivo, hepatitis-C virus progeny emerge from the endoplasmic reticulum of the cell as enveloped virions (i.e. lipid-membrane-enveloped virus particles) associated with very low density lipoprotein (VLDL) in the form of a lipoviral particle′. In order to infect a new cell, the particle may have to bind to cell surface receptors (including tetraspanin, scavenger receptor-B1 and LDL-receptor), SRB 1 and LDL-R are lipoprotein receptors. The receptors are associated with ‘lipid rafts’ (membrane microdomains that are enriched with cholesterol and saturated glycosphingolipids). Once inside the endosome of the cell, the virus may have to further interact with a cholesterol receptor ‘Niemann-Pick type-C disease like protein 1’ (NPCL1) in order to escape into the cytoplasm (Sainz, Barretto et al. 2012). Once inside the cytoplasm of the cell, the virus subverts the lipid metabolism of the endoplasmic reticulum to create its own organelle, the ‘membranous web’, to support the function of its own replicative apparatus. Assembly of virions occurs on the lipid droplet (the immediate precursor of VLDL in the ER), initiated by the binding of core protein to the surface of the droplet. The intact virions then emerge as lipoviral particles associated with VLDL and the whole cycle repeats.

The inventors hypothesize that HCV, because it manipulates and exploits so many aspects of hepatocyte lipid metabolism for its own replication, is bound to have specific and measurable effects on the lipid composition of the cell, and to realize moreover, that these changes will be manifest in the blood in the form of altered lipid composition of blood lipoproteins secreted by the liver (particularly components of VLDL), as well as being accessible to analysis as changes in the lipid composition of liver biopsy specimens. In addition, the inventors have realized that the ‘lipidomic imprint’ of HCV on infected cells, being a measure of the effect of the virus on its host cell, i.e. its impact on liver lipid metabolism, may be a better marker of disease activity than is viraemia, since it may more directly reflect the adverse effects of the virus on liver function and pathology, and since it represents the summation of a complex cellular metabolic response to virus infection, which will likely be influenced by multiple gene polymorphisms—each having a minor contribution, and being of limited predictive value individually. Furthermore, the inventors have recognized that the prognostic value of lipidomic signatures in plasma and biopsy specimens of hepatitis-C infected patients, represent a so-far untapped resource of biomarkers, which can be used in concert with genetic polymorphisms and proteomic biomarkers to achieve enhanced predictive accuracy of response to particular treatment regimens, and the risk and rate of development of fibrosis, cirrhosis and hepatocellular carcinoma. The inventors have also recognized that hepatocellular carcinoma itself, being derived from liver cells which are very active in lipid metabolism, will have its own characteristic lipidomic signature—reflecting changes in lipid metabolism characteristic of the transformed state of the hepatocyte, and that signatures of hepatocellular carcinoma in the form of blood lipoprotein lipid composition can be used for the early detection of liver cancer, which is currently unreliable with existing biomarkers such as alpha-foetoprotein, which is not universally expressed by HCC (expressed in about 80% of cases (Huo, Hsia et al. 2007)). Accordingly, the inventors have studied the effects of infection with replication-competent HCVcc upon the lipidome of hepatocellular carcinoma cells (Huh7.5), and the lipidomic composition and lipidomic response of uninfected and infected cells to iminosugar drugs which are inhibitors of ER alpha-glucosidases and of glucosylceramide synthase, and which have known effects on protein folding (via glucosidase inhibition) (Branza-Nichita, Durantel et al. 2001; Chapel, Garcia et al. 2006; Chapel, Garcia et al. 2007) and/or upon lipid metabolism via inhibition of glucosylceramide synthase (Platt, Reinkensmeier et al. 1997; Butters, Dwek et al. 2003; Butters, Dwek et al. 2005), and by inhibition of beta-glucosidase-2 (GBA2)—a neutral extra-lysosomal glucosylceramidase (Boot, Verhoek et al. 2007).

### Fatty Acid Content of Infected and Uninfected Cells

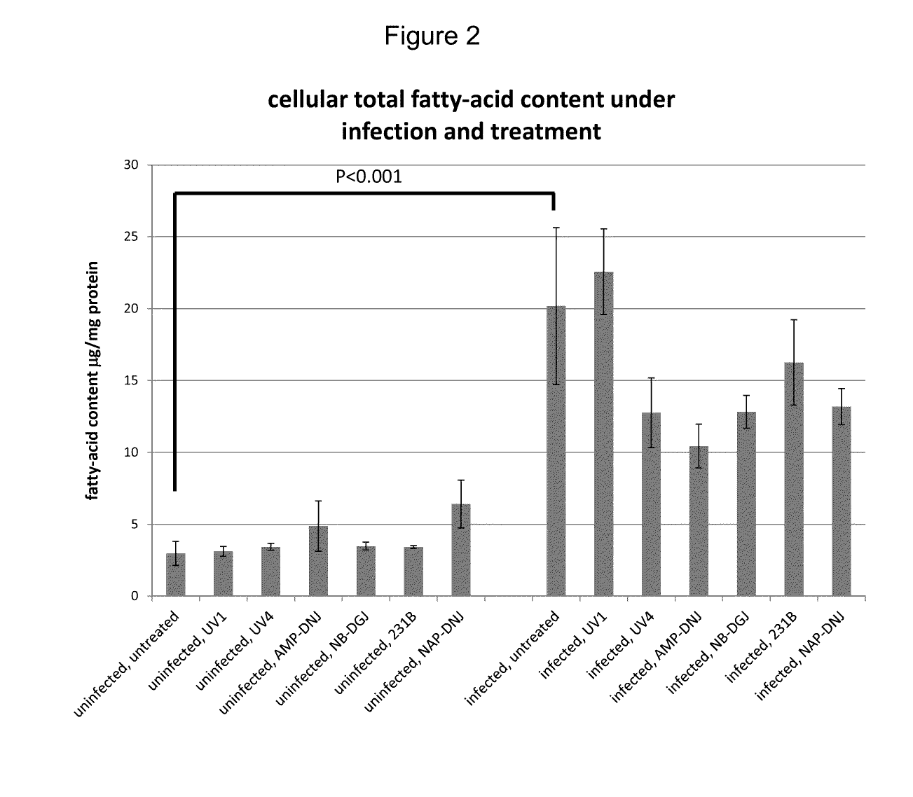

The total fatty acid content (free plus lipidic fatty acyl chains) of hepatoma cells in the uninfected and infected state was measured (FIG. 2). Infected cells were much higher in their fat content (3-5 fold), but the reasons for this elevation are not immediately obvious. For example, the cells can import fats (via lipoprotein receptors); likewise they can export them (as lipoproteins) and also can make them afresh by de novo lipogenesis. Reasons are adduced below to suggest that the elevated fat content of infected cells involves import of essential fatty acids (presumably as the lipid components of lipoproteins), which these human cells cannot make, and moreover, that the high fat content is not likely to be produced by de novo lipogenesis which is heavily suppressed in the infected state. Reduced lipoprotein export (a known feature of HCV infection in liver cells) is another possible explanation for these observations.

### Global Cellular Fatty Acid Composition of Uninfected Cells

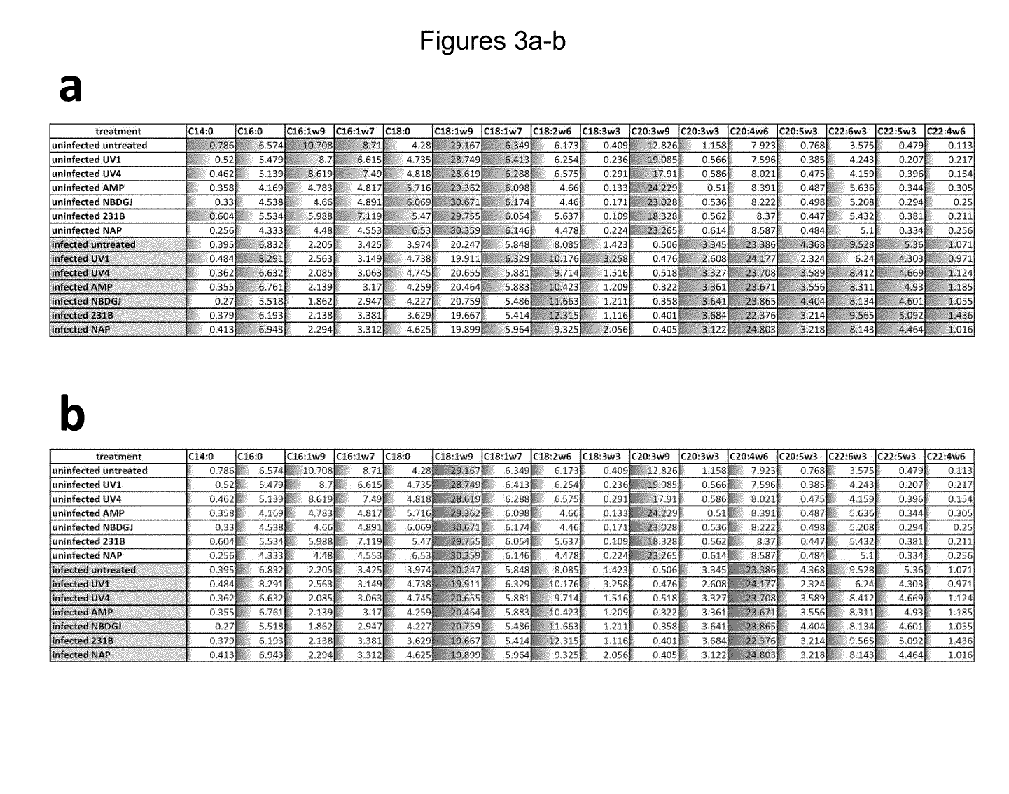

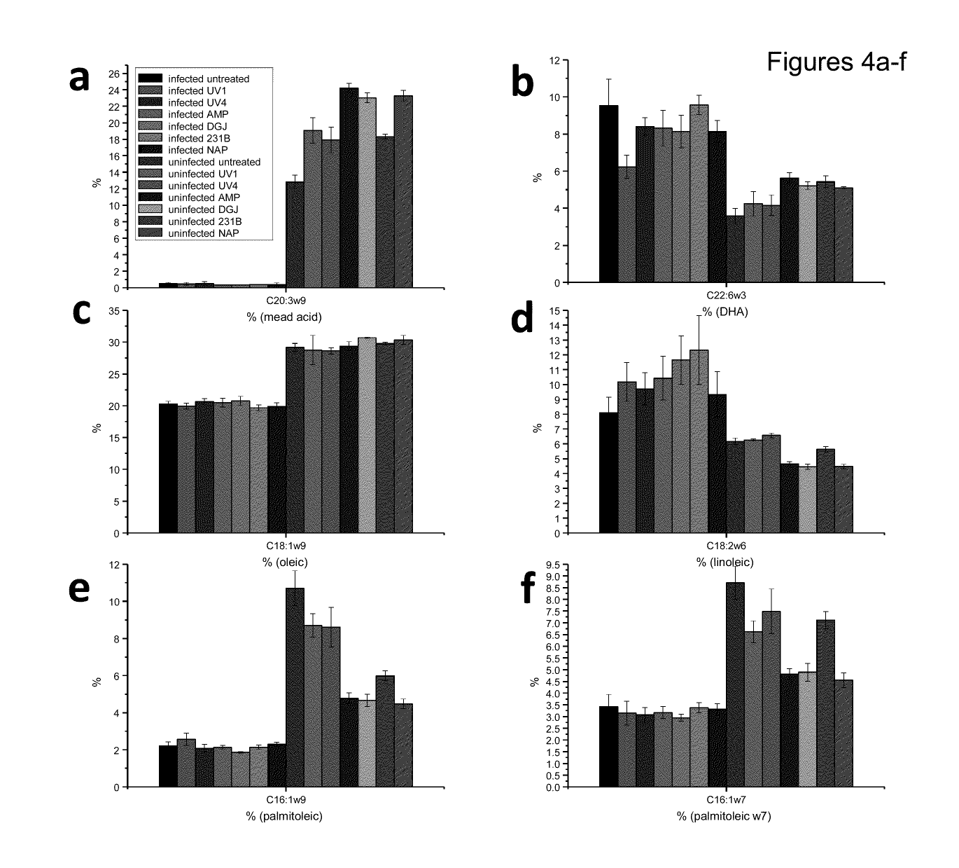

In order to gain an overall impression of the effects of infection and iminosugars on the lipidome of the host cells, the total fatty acid composition of uninfected cells (as fatty acid methyl esters) after acidic transmethylation of total lipid extracts was first examined. This analysis includes non-esterified and esterified fatty acids (the latter comprising parts of cholesterol esters, triglycerides and various phospholipids as well as sphingolipids) FIG. 3. It was surprising to discover a very high content of ‘Mead acid’ (20:3 omega-9, accounting for 13% of total fatty acids) in untreated, uninfected host cells (FIG. 4). Mead acid is produced from palmitate (C16:0), the immediate product of de novo lipogenesis, by further reactions of chain elongation and desaturation. Primary liver cells express much lower levels of Mead acid as a percentage of their fatty acid profile than are seen here for the cultured Huh7.5 hepatoma cells (Claude Wolf, personal communication).

Mead acid is grossly elevated by conditions of essential fatty acid deprivation in vivo in man and animals (Siguel, Chee et al. 1987; Duffin, Obukowicz et al. 2000). Elevation of Mead acid therefore indicates that the uninfected host cells were effectively deprived of essential fatty acids, namely linoleic acid (18:2 omega-6) and alpha-linolenic and (18:3 omega-3) which are the predominant dietary essential fatty acids (being required for the synthesis of the highly polyunsaturated fatty acids, including omega-6 arachidonic acid and omega-3 docosahexaenoic acid).

This observation may suggest that hepatoma cells in HCC patients, unlike healthy liver cells, may secrete Mead acid as the fatty acyl chains of lipid elements of lipoproteins such as VLDL, and moreover that such changes in VLDL composition may be maintained in the face of dietary fluctuations in essential fatty acids. Unlike alfa-foetoprotein, which is a clinically useful protein biomarker of heptatocellular carcinoma, the inventors hypothesize that these changes in VLDL composition may manifest more universally in HCC patients, independently of whether they are positive for serum alfa-foetoprotein (the classical biomarker for HCC). As such, the inventors hypothesize that a diagnostic test based on elevated Mead acid in VLDL may be a more sensitive and reliable indicator of underlying hepatocellular carcinoma than is alfa-foetoprotein (which is elevated in a subset of about 80% of patients), and that such a test may be complementary to at least one another test in the diagnosis of HCC providing synergistic or added value in terms of accuracy and reliability of the diagnosis.

### Global Fatty Acid Composition of HCVcc Infected Cells

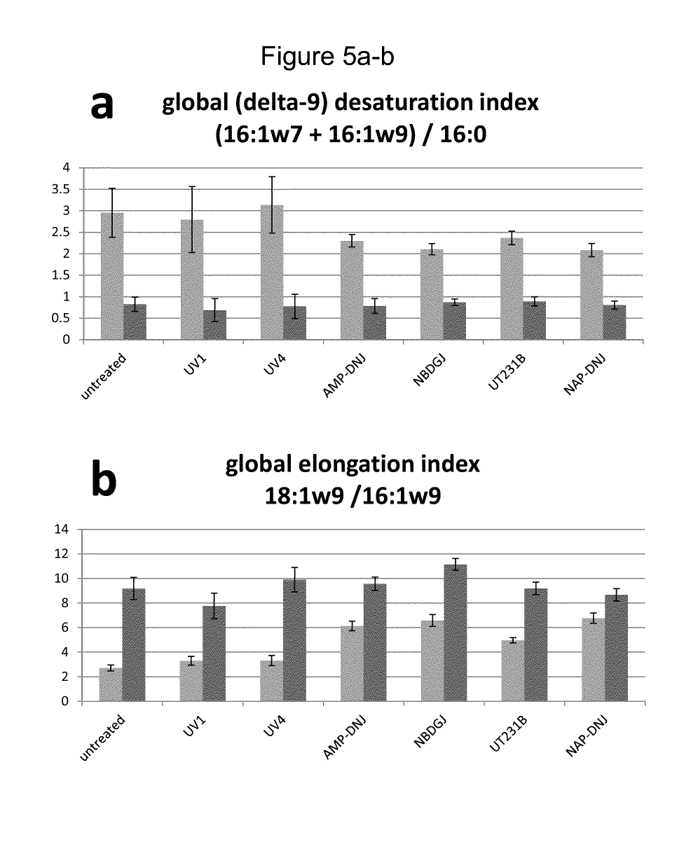

Infection with HCV reduced the cellular content of Mead acid dramatically (over 20-fold, FIG. 4). It also reduced the abundance of other non-essential fatty acid by-products of de novo lipogenesis, namely palmitoleic (C16:1 w9 and w7), and oleic (C18:1 w9) acids (compared to the uninfected state). However, vaccenic acid (C18:1 omega-7), another non-essential fatty acid produced from de novo lipogenesis, was not altered. The mechanism by which HCV reduces the abundance of Mead acid and the other by-products of de novo lipogenesis, or the reason for this effect (perhaps a self-protective reaction of cellular metabolism), are not known with certainty, however the availability of palmitate (16:0) the immediate product of de novo lipogenesis, would not appear to be a limiting factor since the compositional abundance of this fatty acid was not altered by infection. This may suggest, specifically, that HCV may inhibit the further desaturation and chain elongation required to reach Mead acid. (Mead acid is produced in human cells from palmitic acid (16:0) by successive steps of desaturation and elongation involving delta-9 desaturase, delta-6 and delta-5 desaturases, and elongases ELOVL6 and ELOVL5). Notably, dietary cholesterol in the rat has been observed to suppress the activity of delta-5 and delta-6 desaturases in liver (both of which are needed for Mead acid synthesis) (Mariana, Vazquez et al. 1992; Bernasconi, Garda et al. 2000). Although the present invention is not limited by its theory of operation, suppression of Mead acid synthesis could therefore be a consequence of elevation of cellular cholesterol by HCV infection: e.g. HCV is known to elevate cellular cholesterol (Sagan, Rouleau et al. 2006; Kapadia, Barth et al. 2007; Waris, Felmlee et al. 2007; Ye 2007). The reduced desaturation of non-essential (endogenously synthesized) fatty acids seen in the infected state may also have originated from the effects of highly polyunsaturated essential fatty acids such as arachidonic and docosahexaenoic acid, which were (surprisingly) elevated by infection (see later) and which are known to inhibit the expression of all three relevant desaturases (delta-9, delta-6 and delta-5) in liver (Cho, Nakamura et al. 1999; Cho, Nakamura et al. 1999; Ntambi 1999). It was observed that the delta-9 desaturation index was decreased in infected cells (FIG. 5), consistent with elevated PUFA or cholesterol in the infected state.

### Reduced Desaturation and Increased Elongation of Non-Essential Fatty Acids in the Infected State

In principle, the reduction of Mead acid in the infected state could be a consequence either of reduced desaturase enzyme activities as noted above, or of reduced elongation, since both classes of enzyme are required for its synthesis. However, fatty acid elongation was not decreased by infection, rather there was evidence to the contrary (in the global fatty acid profile) of increased elongation of fatty acids in the infected state as assessed by the ratio of (18:1 omega-7/16:1 omega-7) (FIG. 5), a function of the combined activities of ELOVL-1 and ELOVL-6. In contrast, there was reduced desaturase activity in the infected state. Thus, delta-9 desaturase (also known as stearoyl-CoA desaturase-1, SCD1) desaturates both palmitic (16:0) and stearic (18:0) acids. The 16:1/16:0 ratio was decreased by infection, suggesting a decrease of delta-9 desaturase activity: i.e. the activity of delta-9 desaturase was assessed as a ratio of the abundance of palmitoleic acids over palmitic acid species ((16:1 omega-7+16.1 omega-9)/16:0) which was found to be decreased in the infected state. This analysis may indicate a reduced activity of delta-9 desaturase in the infected state, combined with increased elongation. These findings (relating to delta-9 desaturase) concur with the reduced levels of Mead acid indicating reduced activity of desaturases-6 and 5, such that reduced desaturase activity (delta-9, delta-6 and delta-5) is a likely reason for the reduced levels of non-essential unsaturated fatty acids (including Mead acid) in the infected state.

### The Effect of Iminosugars on Global Fatty Acid Composition

In the uninfected state, the iminosugars, in general, were found to increase the already high Mead acid component of the global fatty acid composition at antiviral concentrations (FIG. 4). In the case of one of the iminosugar compounds (AMP-DNJ), Mead acid content was almost doubled. These effects of the iminosugars were statistically significant.

The stimulation of Mead acid production by iminosugars appears to be a consequence of further-increased activity of desaturase enzymes delta-6 and delta-5, i.e. over and above the constitutively high levels in these cultured cells as evident from their already high Mead acid content. This effect of the iminosugars was strikingly opposite to that of HCV infection but, paradoxically, was not detectable in the infected state, which was very much dominated by the effects of the virus. This effect of the iminosugars in the uninfected state may indicate an increased sensitivity to insulin brought on by iminosugar treatment of the cells. Thus, one of these compounds (AMP-DNJ, also known as AMP-DNM), improves hepatic insulin sensitivity, decreases fatty acid synthase activity and abolishes hepatic steatosis in obese mice (Bijl, Sokolovic et al. 2009). Moreover, insulin stimulates delta-6 desaturase expression and activity (which is rate-limiting for Mead acid synthesis (Wang, Botolin et al. 2006), which may support the hypothesis that the iminosugars are increasing insulin sensitivity. The inventors hypothesize that the observation that type-II diabetes is a negative prognostic indicator for treatment response (to interferon+ribavirin) in HCV infected subjects, and that patients cured of HCV infection are also cured of insulin resistance (Clement, Pascarella et al. 2009; Eslam, Khattab et al. 2011) may suggest that the insulin-sensitizing effect of the iminosugars may be advantageous in antiviral treatment with iminosugar drugs by counteracting an underlying metabolic defect in hepatocytes that favors replication of the virus.

### Effects of HCV Infection Upon the Essential Polyunsaturated Fatty-Acid Component of the Global Fatty Acid Composition

The essential fatty acids linoleic and alpha linolenic acid cannot be synthesized by mammalian cells. Moreover, it was discovered (above) that the cells are very deficient in these essential fatty acids. Thus, it was surprising to find markedly increased abundance of highly polyunsaturated omega-6 and omega-3 fatty acids such as arachidonic and docosahexaenoic acid in the infected state. Although the present invention is not limited by its theory of operation, the increased relative abundance of the these highly polyunsaturated species in the infected state may simply reflect the fact that they are no longer being diluted by endogenously synthesized fatty acids from de novo lipogenesis, which is suppressed by the virus.

Given that polyunsaturated fatty acids (PUFA), such as docosahexaenoic acid, in the free state, are antiviral vs. HCV in both replicon and infectious virus systems (Leu, Lin et al. 2004; Kapadia and Chisari 2005; Miyoshi, Moriya et al. 2011), the high content of such PUFA in the infected cells is all the more surprising. Although the present invention is not limited by its theory of operation, it is possible that the high omega-3 and omega-6 PUFA content of cholesterol esters and triglycerides in the infected state (see below) may reflect a tendency of the virus to favor sequestration of these fatty acids into the lipid droplet, where their ability to inhibit viral replication is limited.

The surprising elevation of cellular omega-3 and omega-6 highly polyunsaturated fatty acids observed in the infected state, may indicate that elevated plasma levels of these fatty acids could indicate quantitatively the extent of HCV infection, or the metabolic impact of HCV infection upon liver function, however, these essential fatty acids (EFA) are common dietary components (abundant in meat (omega-6) and abundant in fish (omega-3)), such that a biomarker strategy based on abundance of these fatty acid markers in the global plasma fatty-acid profile may be easily confounded by changes in diet. A more sophisticated analytical approach may be needed, mindful of the potential confounding effects of diet on the total lipid fatty acid profile.

### Fatty Acid Composition of Individual Lipid Classes

Cholesterol Esters

The most common fatty acid species of cholesterol esters were measured in both the uninfected and infected state.

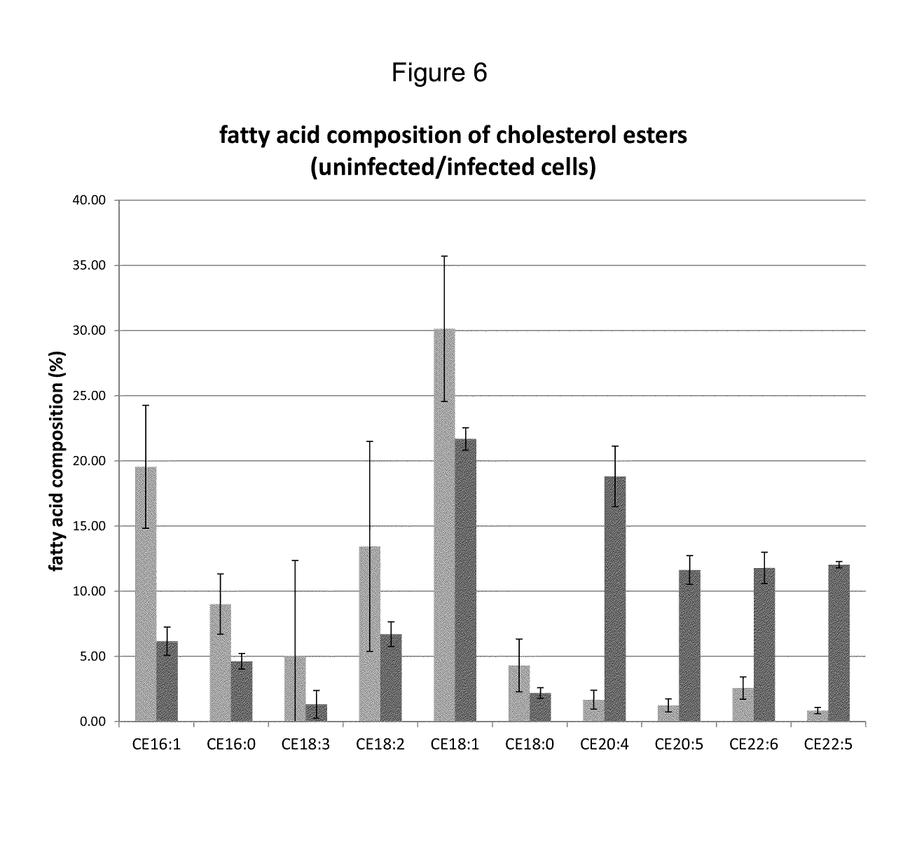

Infection brought about marked changes in the fatty acid composition of cholesterol esters (FIG. 6), with substantial increases in essential omega-3 and omega-6 fatty acids (20:4, 20:5, 22:6 and 22:5) of 5-14 fold, and decreases in monounsaturated species (16:1, 18:1) which do not convert to the EFA forms: i.e. cholesterol esters reflect similar changes to those wrought by infection on the global fatty acid profile, and may therefore have been a significant contributor to the global profile of the cultured hepatoma cells. Unlike the global profile, no strong differences were apparent in the fatty acid profile of cholesterol esters under the influence of iminosugars, either in the infected or in the uninfected state, although Mead acid (not normally found as a significant component in cholesterol esters) was not measured in the cholesterol ester fatty acid profile analysis. Since cholesterol esters are major components of the lipid droplet, which is the immediate precursor of very low density lipoprotein (VLDL), and since VLDL is the lipoprotein component of the HCV lipoviral particle, it is apparent from this insight that the elevation of cholesterol esters enriched for EFA species could be a useful biomarker for hepatitis-C virus infection, or for assessing the metabolic impact of HCV infection upon liver cells in infected patients.

Triglycerides

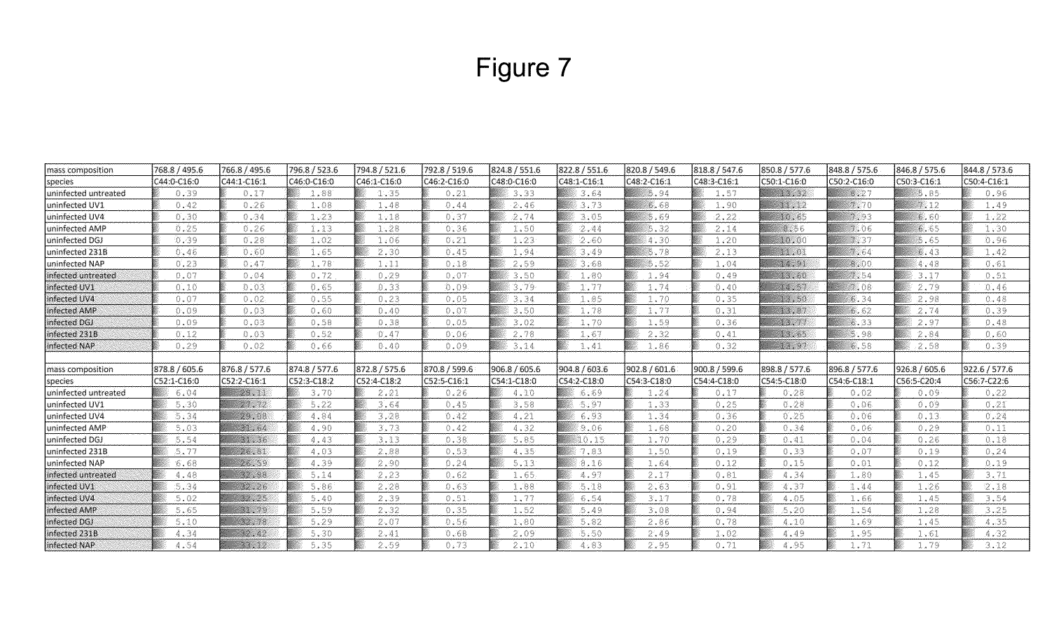

Triglycerides form the major component of the lipid droplet, which forms (along with cholesterol esters) the core of secreted VLDL and lipoviral particles. Given our rationale developed here that blood VLDL/lipoviral lipid composition is likely to be a sensitive indicator of the effect of HCV infection on hepatocyte metabolism, triglycerides are therefore of particular interest. Unlike cholesterol esters, where there is only one fatty acyl chain per molecule, in the case of triglycerides there are three, and the three positions are not equivalent with respect to the fatty acyl chains which tend to be found in each position, reflecting substrate preferences of synthetic enzymes as well as the availability of free fatty acid precursors in the cell (Berry 2009). These features of triglyceride biosynthesis provide more degrees of freedom for a greater diversity of fatty acyl isomeric compositions than for other lipid classes. FIG. 7 shows the percent composition of the different triglyceride species found and the influence of infection and of iminosugars upon this composition.

Notably, the compositional abundance of the most abundant species of triglyceride ‘C52:2-C16:1’ containing palmitoleic acid (16:1), representing one quarter to one third of all triglyceride species, was virtually unchanged by infection. However, there were very marked changes in some of the more minor triglyceride species. Thus, nine triglyceride species were reduced in abundance >2 fold, whereas six triglyceride species were increased in abundance >2 fold. The biggest changes were in the following triglyceride species which were increased upon infection >15-fold:—

C54:5-C18:0

C54:6-C18:1

C56:5-C20:4

C56:7-C22:6

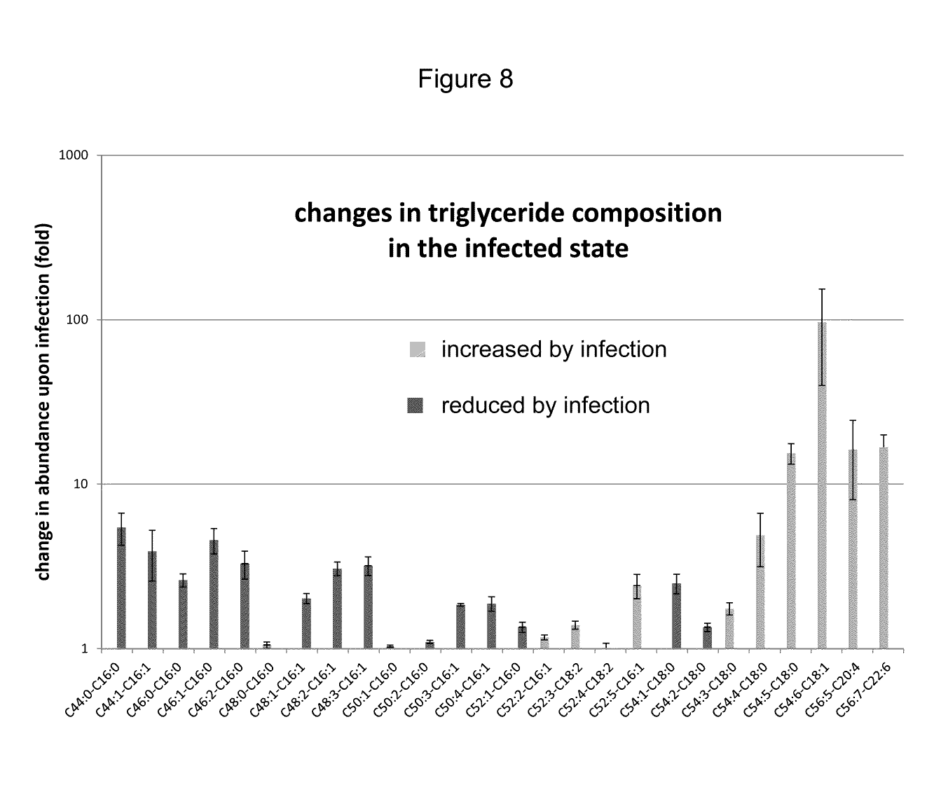

Notably, C54:6-C18:1, was increased 96-fold by infection, although it still represented only 1.7% of the triglyceride composition in the infected state. Changes such as these may not be apparent in traditional blood analysis of triglycerides as used in routine clinical diagnostic tests, because these tests measure total triglyceride and do not break down triglyceride species according to their fatty acid composition or molecular species. Notably also, species containing very high numbers of double bonds and explicitly containing essential fatty acids, C56:5-C20:4 (containing arachidonic acid 20:4), and C56:7-C22:6 (containing docosahexaenoic acid) were highly elevated in the infected state (>16-fold), although still comprising only minor components of the total cellular triglyceride pool (FIG. 8).

The entirely saturated species C44:0-C16:0 was reduced five-fold in the infected state, such that reduction in the abundance of this triglyceride species in blood triglycerides or VLDL could be useful in determining the effects of hepatitis-C virus upon liver lipid metabolism. The iminosugar compounds had no systematic effect upon triglyceride fatty acid composition, except for a tendency to increase the abundance of this saturated species in the uninfected state and paradoxically, to reduce the abundance of this species in the infected state. The minor effects of iminosugars on this particular triglyceride species are not expected to be of particular diagnostic or prognostic significance.

Phosphatidylcholine

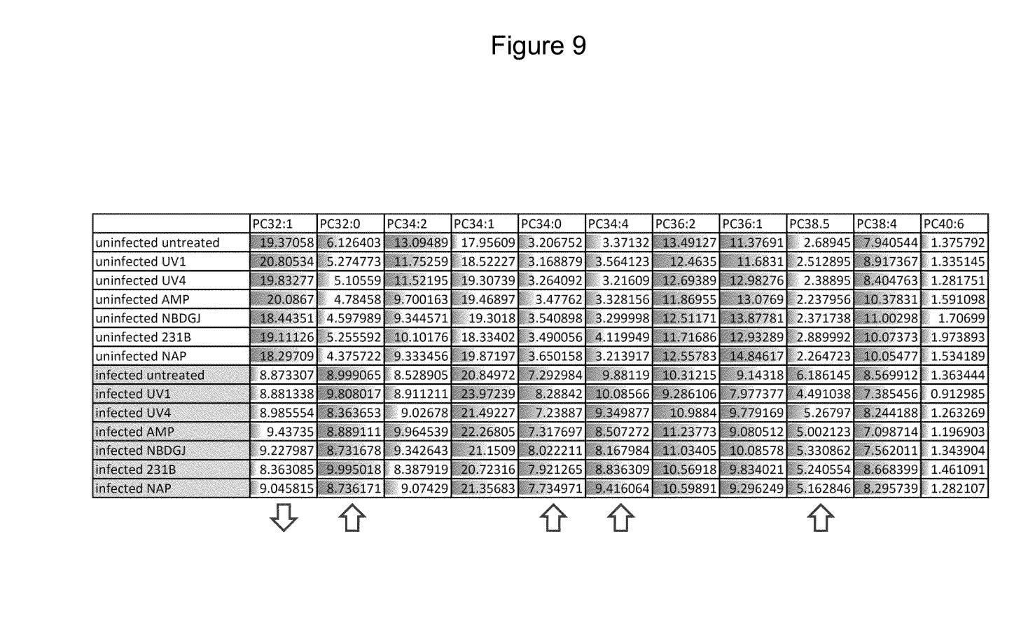

In contrast to the ether bonded form of PC (see below), the fatty acid composition of the ester-bonded phospholipids PC, PE, PS and PI were extensively remodeled by infection, as was the lyso form of PC. In the case of the PC diester form, the level of the monounsaturated PC32:1 species was decreased while saturated PC32:0 and 34:0 and PUFA-enriched PCs (PC34:4 and PC38:5) were elevated by infection (FIG. 9). It is not possible to distinguish explicitly Mead acid or other individual polyunsaturated fatty acids in this analysis due to isomeric ambiguity of the whole molecular species. Nevertheless, the increase in polyunsaturated fatty acyl forms of PC observed in the infected state concurs with and confirms the surprising results of the analyses above on global fatty acid profile and cholesterol ester profile—namely increased abundance of essential polyunsaturated fatty acyl species in the infected state with incorporation of these unsaturated forms into membrane phospholipid.

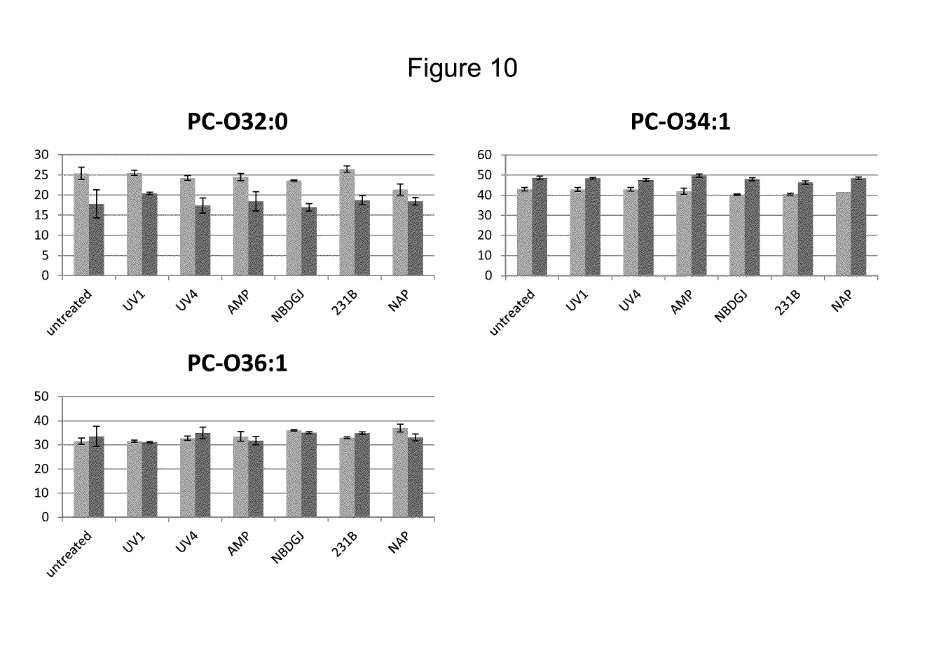

The fatty acid composition of the ether-phospholipid form of PC (i.e. the ‘plasmalogen’ form synthesized in the peroxisome) was virtually unaltered by infection (FIG. 10). Selective remodeling of ester-bonded phospholipids, made in the ER, indicates that HCV has a compartment-specific effect in the remodeling of phospholipids affecting the ER but not the peroxisome, consistent with its intimate dependence on the ER for the genesis of the ‘membranous web’ (noted earlier) its core protein assembly on the lipid droplet (in close association with the ER) and its budding from the ER as a lipoviral particle.

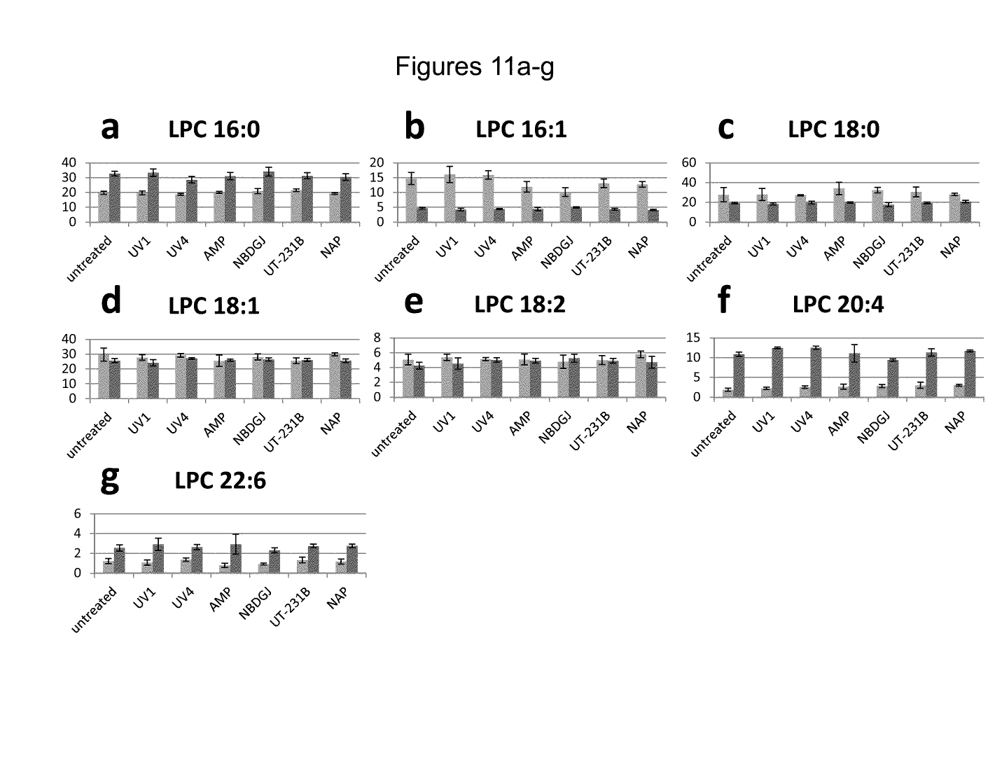

The analysis of lyso-PC (FIG. 11) provides an opportunity to resolve the isomeric ambiguity noted above. In the case of lyso-PC, derived from diester PC, but having only one fatty acyl chain, there was a reduction in unsaturated palmitic acid in the infected state (16:1) with a reciprocal elevation in 16:0, consistent with observations above on the delta-9 desaturation index and demonstrating that these prevailing changes in the global profile are also reflected in membrane as well as storage lipids. Likewise for lyso-PC there was explicit elevation in 20:4 (arachidonic acid), and 22:6 demonstrating that these essential fatty acids, more abundant in the infected state, also found their way into membrane phospholipids.

Phosphatidylethanolamine

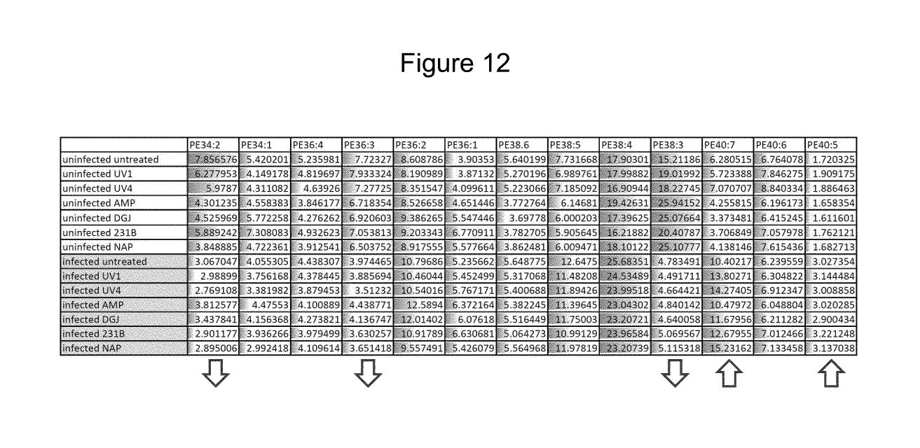

In the case of the analysis of the diester form of PE (FIG. 12), Mead acid (C20:3 omega-9) was identified explicitly. As per the global fatty acid profile, infection brought about a >20 fold reduction in Mead acid, whereas (in the uninfected state only) the iminosugar treatments give rise to increased levels of Mead acid—up to two-fold depending on the iminosugar compound. Likewise, infection brought about a marked decrease in palmitoleic acids (16:1 omega-7 and omega-9) and a marked increase in the essential omega-3 and omega-6 fatty acids (20:3 omega-3; 20:4 omega-6; 20:5 omega-3; 22:6 omega-3; 22:5 omega-3; and 22:4 omega-6). These findings of the fatty acyl composition of PE strongly confirm the findings of the fatty acid global profile, the cholesterol ester profile and the diester-PC profile. Unlike the diester PC profile however, in the case of PE there was no balancing elevation in the abundance of saturated forms (compared to monounsaturated), which may suggest that, in the case of PE, infection may have a major influence on its membrane fusion and fission properties (e.g. required for cell division, viral budding from the ER, etc.).

Phosphatidylserine

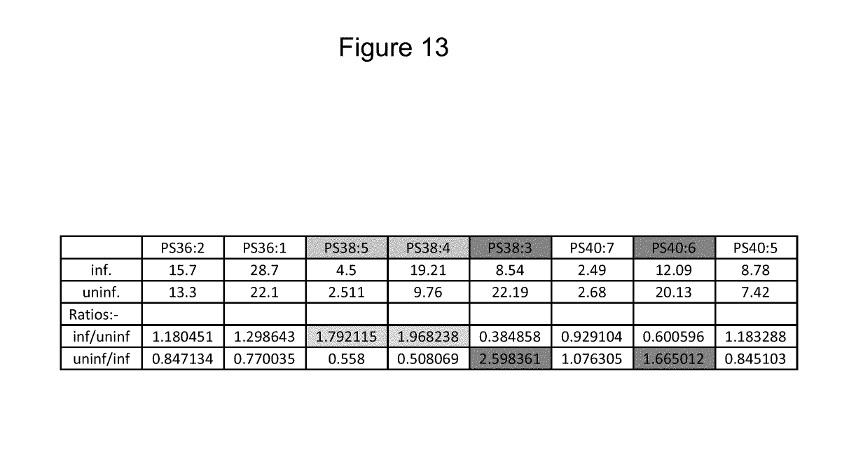

In the case of the diester form of PS, infection brought about a >2-fold reduction in the 38:3 species, presumed to comprise 18:0 and Mead acid (20:3 omega-9), indicating a lesser plasticity to the prevailing changes in the global fatty acid profile than for the other lipid classes (FIG. 13). Lesser plasticity might reflect a lower turnover rate of PS. Furthermore, the 40:6 species was reduced by infection. However, it is not clear at this level of breakdown whether this PS species represents a species containing a single 22:6 paired with a C18:0, or a pair of C20:3.

Phosphatidylionositol

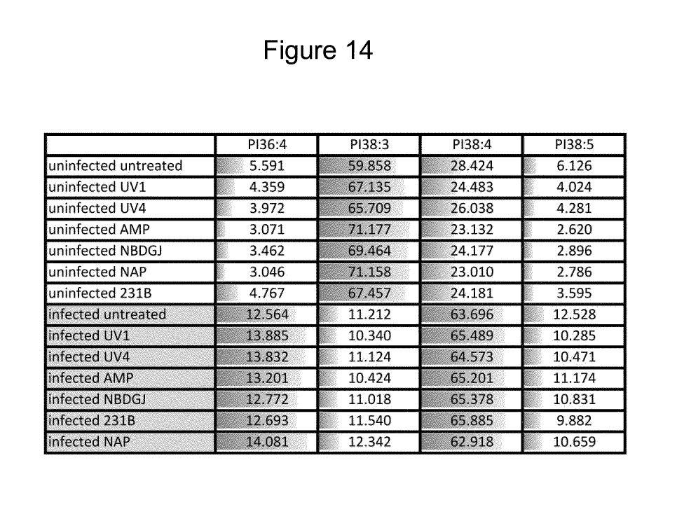

The diester form of PI, having only four molecular species, showed marked changes in fatty acid composition upon infection (FIG. 14). Infection was associated with a decreased level of PI38:3 of 6-fold, and an increased proportion of the remaining PUFA-enriched species of PI (PI36:4, 38:4 and 38:5) of about two-fold in each case. The biggest shift in PI composition was caused by infection (as distinct from iminosugar treatment) which resulted in substantial replacement of the 38:3 species by 38:4. Because the presumed structure of PI38:3 is 18:0/20:3, the changes upon infection are consistent with a decreased availability of Mead acid (20:3 omega-9) in the infected state, which is replaced with arachidonic acid (20:4 omega-6) which is the most abundant fatty acid in the infected state: i.e. a change from 18:0/20:3 (stearic/Mead) by 18:0/20:4 (stearic/arachidonic). In the uninfected state only, there was a tendency towards increased levels of the PI38:3 species in the presence of iminosugar compounds, weakly reflecting the strong elevation of Mead acid by iminosugars seen in the global fatty acid profile. PI is prominently involved in intracellular signaling mechanisms, and changes in PI fatty acid composition (brought about by infection or iminosugar treatment) might be expected to influence PI mediated intraceullular signaling (e.g. affecting insulin sensitivity). Notably, arachidonic acid (omega-6) blocks the activation of PI3 kinase by insulin, preventing its induction of glucose-6-phosphate dehydrogenase via P38 MAP kinase (Talukdar, Szeszel-Fedorowicz et al. 2005). Also, changes in the fatty acid composition of PI caused by infection may alter the substrate behavior of PI towards PI3 kinase. Furthermore, there are additional reasons (distinct from insulin resistance) to believe that this change in PI composition brought about by infection could be of pathological significance in hepatitis-C virus infection. Thus, PI is a major source of arachidonic acid for ecosaniod synthesis, and the 3-fold increase in PI arachidonic acid in the infected state, may enhance the host inflammatory response to the virus via increased biosynthesis of these bioactive products.

Significance of Phospholipid Fatty-Acid Profiles for Biomarker Identification

The liver secretes HCV virions as part of the lipoviral particle (described above) which comprises the HCV virion associated with a VLDL particle. Although the present invention is not limited by its theory of operation, the inventors hypothesize that because the lipidic surface of VLDL particles comprises predominantly PC and PE, any effects of the virus on cellular fatty acid profile of PC and PE will be reflected as alterations in the fatty acid profile of VLDL in the blood of infected patients. In the case of PC, only changes to the fatty acid profile of the diester form upon infection were observed. It may follow that changes in the diester form of PC in VLDL may be of most interest from the point of view of identifying biomarkers, and that the composition of the ether form may be a useful control. First, in this regard, it is notable that the PC32:1 species was decreased while saturated PC32:0 and 34:0 and PUFA-enriched PCs (PC34:4 and PC38:5) were elevated by infection. Thus decrease of 32:1 combined with elevation of (32:0; 34:0; 34:4 and 38:5) species of diester PC would indicate the activity of HCV upon phospholipid metabolism of the hepatocyte.

In the case of PE, there was elevated Mead acid in the uninfected state, which would be reflected in elevated Mead-acid in hepatocellular carcinoma derived blood VLDL particles, such that blood VLDL Mead acid content would serve as a useful marker of hepatocellular carcinoma. Furthermore, infection brings about a marked decrease in palmitoleic acids (16:1 omega-7 and omega-9) and a marked increase in the essential omega-3 and omega-6 fatty acids (20:3 omega-3; 20:4 omega-6; 20:5 omega-3; 22:6 omega-3; 22:5 omega-3; and 22:4 omega-6). This latter constellation of changes in plasma VLDL characteristic of the infected state may be indicative of the impact of HCV infection upon liver hepatocytes, and may have utility for biomarker purposes.

PI and PS being only minor phospholipid constituents of VLDL, may be less useful as biomarkers of HCV infection. Nevertheless reduced levels of Mead acid in PI in VLDL, or of the 38:3 species of PI may be useful indicators of the impact of infection with HCV. Also, increased levels of PI and PS (normally confined to the intracellular leaflet of the plasma membrane and ER) in VLDL may signify HCV infection. Thus, apoptosis of hepatocytes occurring during HCV infection may result in increasing membrane asymmetry with consequent enrichment of lipids such as PI and PS, normally largely excluded from VLDL, now appearing in greater abundance in the surface phospholipid monolayer of VLDL.

### Abundance and Fatty Acid Composition of Glycosphingolipids in Infected Vs. Uninfected and Treated Vs. Untreated Cells

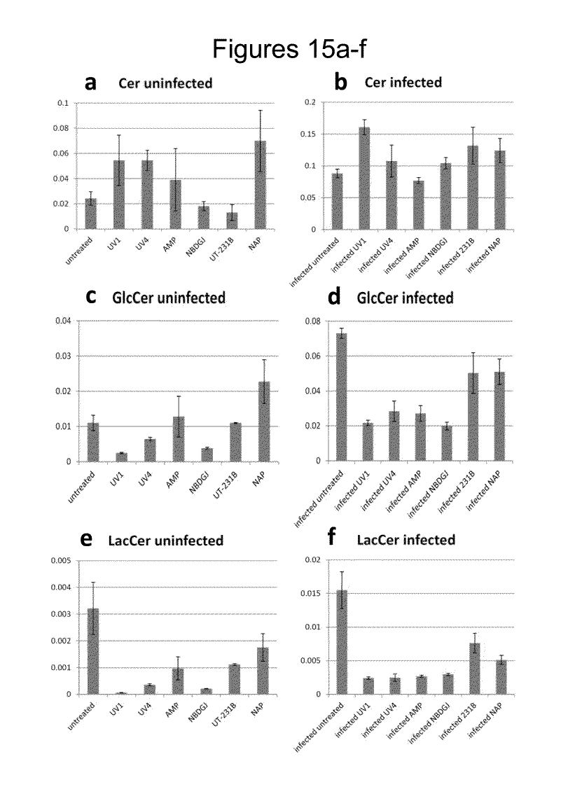

Treatment of infected or uninfected cells with iminosugars at antiviral concentrations strongly reduced the cellular concentrations of ‘glycosylceramide’ (the present mass spectrometric analysis makes no distinction between glucosyl and galactosyl forms), via inhibition of glucosylceramide synthase (FIG. 15). These effects were even more pronounced in the case of lactosyl ceramide, which is an explicit product of glucosyl ceramide (as opposed to ‘glycosylceramide’ which is ambiguous in this mass-spectral analysis with respect to glucose or galactose). These results may have been expected because most of the tested compounds were previously known to be inhibitors of glucosylceramide synthase.

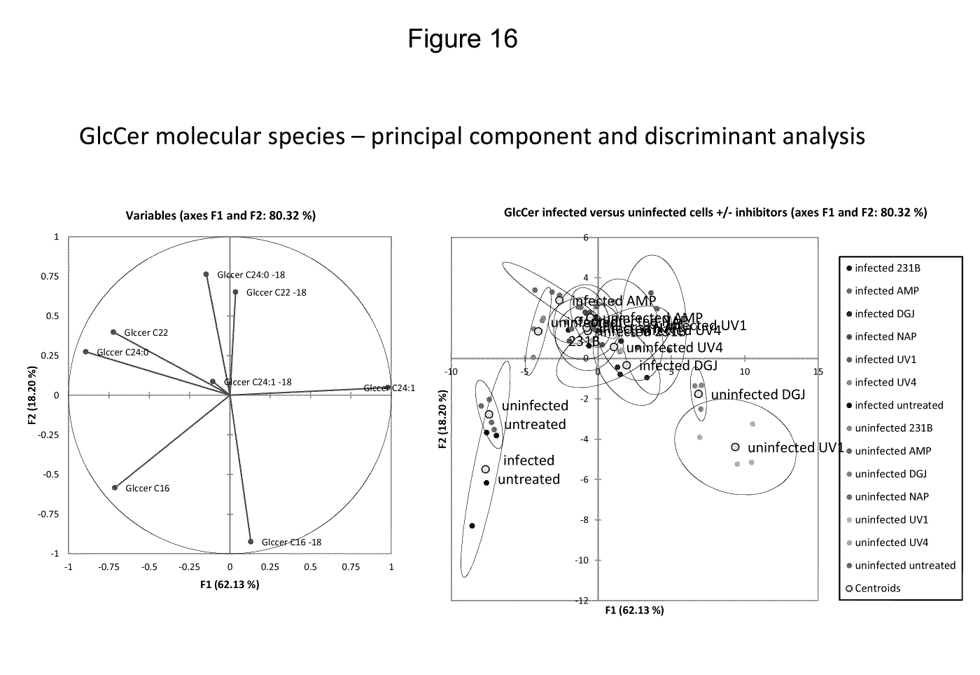

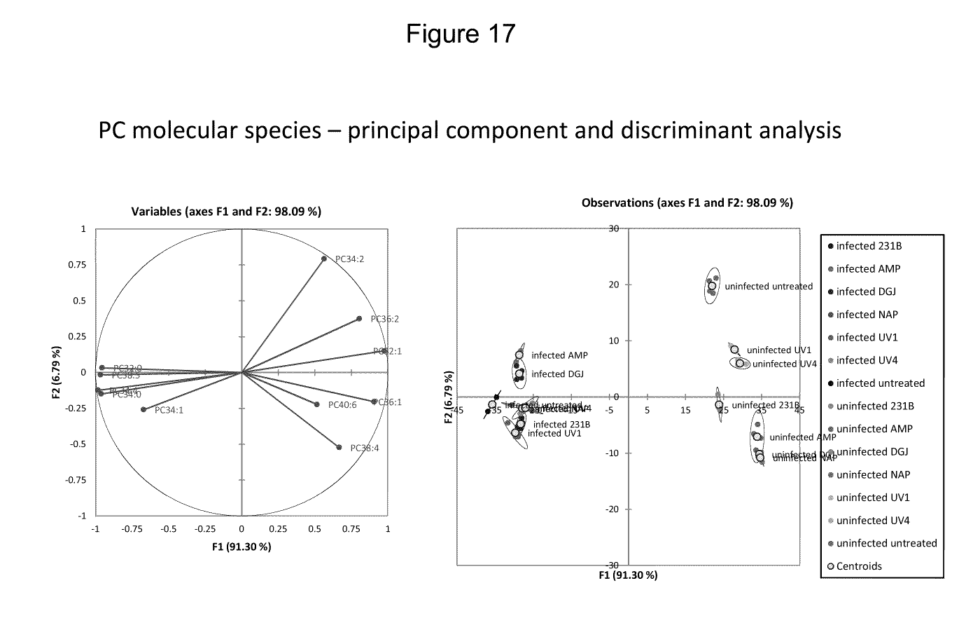

However, unexpectedly, it was also found that the fatty acid composition of glucosyl ceramide was altered by iminosugar treatment, although, surprisingly (given the above observations of extensive remodeling of major and minor cellular lipids by infection, and changes in the delta-9 global desaturation index), the fatty acid composition of GlcCer was not changed by infection. In contrast, iminosugars caused both chain elongation and desaturation of GlcCer (the latter effect being the stronger of the two effects). These phenomena (desaturation and chain elongation) were present in the infected and the uninfected state alike. FIG. 16 describes these drug-responsiveness of glucosylceramide desaturation, and FIG. 17 demonstrates that these changes are specific to sphingolipids (GlcCer and LacCer), i.e. not represented in PC and PE.

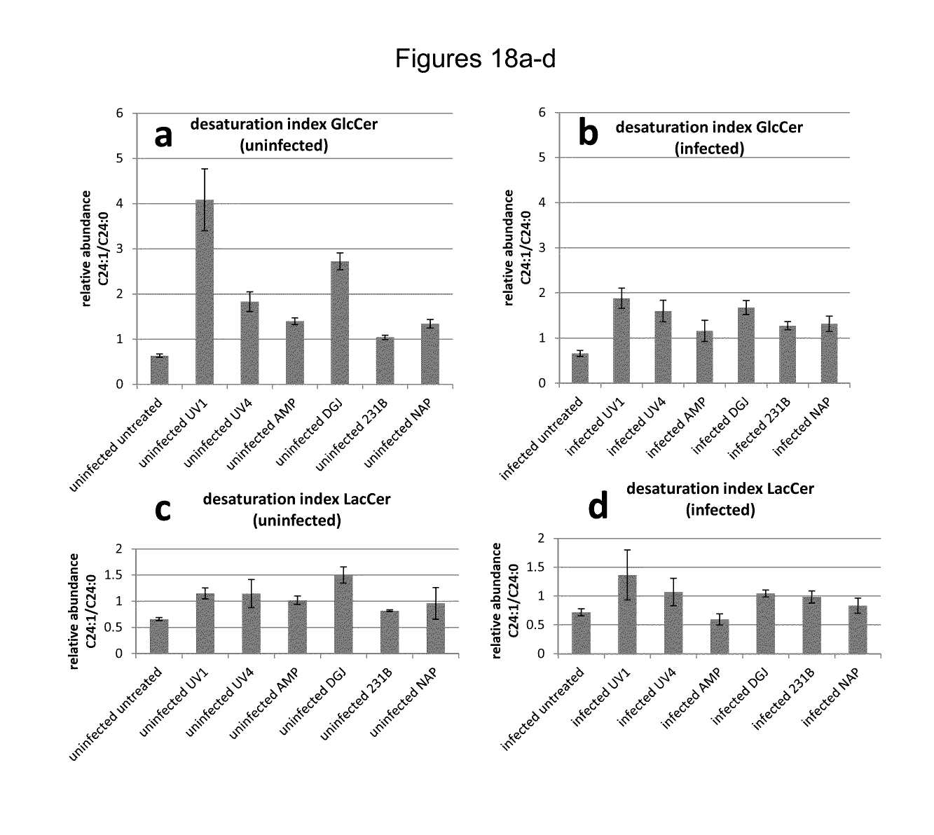

The changes in GlcCer fatty acid composition (increased desaturation in response to iminosugars in the infected and uninfected state alike) may be counter in some respects to the observations and inferences (made earlier) of reduced desaturase activity in the infected state, although, in accord with the observations above, the degree of iminosugar-induced desaturation of GlcCer was less in the infected state. These changes may be conveniently expressed as a ‘desaturation index’ for GlcCer, i.e. ratio of abundance of C24:1/C24:0 (i.e. the molar ratio of nervonic acid/lignoceric acid fatty-acyl chains) (FIG. 18). It was found that the desaturation index of GlcCer and that of LacCer (to a lesser extent) were elevated by iminosugars. In contrast, this index was not changed in the related sphingolipid ceramide (the immediate precursor of GlcCer) (not shown). One may therefore suggest that accumulation of unsaturated GlcCer in the presence of iminosugar inhibitors of GlcCer synthase may represent a preference of the LacCer synthase (galactosyltransferase-I), or later enzymes in the pathway of biosynthesis of more complex glycosphingolipids, for saturated forms, rather than being mediated by an effect of the compounds on delta-9 desaturase activity, which was evidently suppressed in the infected state (as evident from the global fatty-acid profile of the cells, above). These changes might alternatively be expected to result in the synthesis of gangliosides incorporating a greater proportion of unsaturated chains, i.e. when the GlcCer precursor pool of LacCer synthase is very predominantly unsaturated; or to result in interference by unsaturated forms of GlcCer acting as a competitive inhibitors of LacCer synthesis, as outlined below.

Since GlcCer and LacCer are major precursors of gangliosides (Butters, Dwek et al. 2005; Fuller 2010), and because gangliosides are important components of lipid rafts (along with sphingomyelin and cholesterol) (Quinn 2010), the inventors hypothesize that reduction in cellular abundance of GlcCer might be expected to reduce the abundance and/or size of cellular membrane rafts, or to change their functional properties. Moreover, since HCV is highly dependent upon lipid rafts for several stages of its replicative cycle (Aizaki, Lee et al. 2004; Matto, Rice et al. 2004; Aizaki, Morikawa et al. 2008; Weng, Hirata et al. 2010), it may follow that reduced abundance or altered functionality of lipid rafts caused by iminosugars might explain their antiviral effects against HCV. Moreover, it was observed that, in addition to reducing the abundance of GlcCer, iminosugar inhibitors of glucosylceramide synthase also, surprisingly, increase the desaturation of GlcCer, which might also be expected to influence the quantity and properties of lipid rafts: i.e. incorporation of unsaturated gangliosides into lipid rafts (which are characteristically ‘saturated’ microdomains) may alter their structure and function. Since pathological accumulation of gangliosides entraps cholesterol in cellular membranes (as in the ganglioside storage diseases such as Gaucher disease), it may be that the iminosugars may influence cholesterol compartmentalization or trafficking. For example, the depletion of ganglioside components of lipid rafts may liberate cholesterol from rafts increasing its ‘free’ concentration in the membrane. Thus, the iminosugars, in addition to their direct effects on lipid rafts, may mediate their antiviral effect by liberating cholesterol from membrane rafts, as a consequence of changes in the abundance or fatty acid composition of GlcCer.

Liberation of cholesterol from lipid rafts would falsely signify a state of ‘cholesterol overload’ to the cell, causing feedback inhibition of cholesterol synthesis by liberated cholesterol, depriving the virus of the cholesterol it needs for replication.

### Significance of Glycosphingolipid Abundance and Desaturation for Biomarker Purposes

It may follow from the above that the abundance of glucosylceramide and the desaturation index of both glucosylceramide and lactosylceramide in blood lipoproteins may be used as indicators of the effectiveness of antiviral therapy vs. HCV when using iminosugars therapeutically. Likewise, these indices may be used as a measure of response to treatment with inhibitors of glucosylceramide synthase in genetic lysosomal storage disorders such Gaucher and Niemann-Pick type-C disease. Although the abundance of a ganglioside product of glucosylceramide (i.e. leucocyte surface GM3) has been used experimentally as a biomarker for treatment response in Gaucher disease, there has been no suggestion of using the desaturation index of glucosylceramide as a biomarker for treatment response to inhibitors of glucosylceramide synthase in such diseases. Likewise, although the abundance of nervonic acid (24:1) in the global plasma fatty acid profile and in sphingolipids (namely ceramide, sphingomyelin and cerebrosides) is decreased in rat and murine models of type-I diabetes (Fox, Bewley et al. 2011), until now there has been no suggestion that the desaturation index of glucosylceramide may be used as a marker of disease activity or response to insulin sensitizing agents in type-II diabetes. Here the inventors suggest that metabolic syndrome and type-II diabetes may be characterized by elevation of the desaturation index of glucosylceramide in VLDL (due to hyperinsulinaemia), and that treatment with insulin sensitizing agents (such as select iminosugars, biguanides and thiazolidinediones) may reduce this index towards normality, by improving insulin sensitivity (specifically with respect to the glucose-uptake response of the tissues stimulated by insulin, and reduction of glucose production by the liver) and correcting hyperinsulinemia.

Given that metabolic syndrome and type-II diabetes are adverse prognostic indicators for treatment response in HCV with interferon+ribavirin (Clement, Pascarella et al. 2009; Eslam, Khattab et al. 2011), it may also follow that the desaturation index of blood VLDL GlcCer and/or LacCer may be used to identify HCV-infected subjects who are unlikely to respond to interferon+ribavirin. For example, an HCV-infected patient presenting with an abnormally high desaturation index of GlcCer or LacCer expressed as the ratio of 24:1/24:0 (e.g. having hyperinsulinaemia due to undiagnosed metabolic syndrome) may be less-likely to respond to interferon+ribavirin, and may require more aggressive treatment with newly licensed drugs (either alone, or in combination with each other or with interferon+ribavirin). Likewise, such a patient would be more likely than other HCV infected patients to respond to therapy with an iminosugar inhibitor of glucosylceramide synthase, which would reduce hyperinsulinaemia by improving insulin sensitivity in the tissues (including liver). By making sure that hepatitis-C patients receive drugs that they are more likely to respond to, patients may benefit and the cost of treatment may be reduced.

Although the desaturation index of palmitoleic 16:1/16:0, in blood VLDL triglycerides, has been advocated as a marker of metabolic disease (Peter, Cegan et al. 2009), i.e. is elevated in metabolic syndrome, this marker strategy does not anticipate the particular value of the glucosylceramide desaturation index identified here, which is especially relevant to insulin sensitivity by dint of the insulin-sensitizing effects of the iminosugars exemplified by the adamantly compound AMP-DNJ/AMP-DNM, which demonstrates the involvement of glycosphingolipids in the regulation of insulin signaling. Moreover, the present results may indicate that the 16:1/16:0 ratio (unlike the desaturation index of glucosylceramide) would tend to be reduced in infected cells, where HCV infection reduces this ratio (at least in the context of the global fatty acid profile of infected cells) limiting its usefulness as a marker of metabolic disease in the context of HCV infection.

### Implementation of the Diagnostic Test

Blood contains several different lipoprotein forms, some of which vary dynamically over time following ingestion of food. For example, fats are absorbed in the form of chylomicrons which are highest following a meal and which disappear quite rapidly postprandially (within six hours). The chylomicrons contain predominantly dietary fats and comprise triglycerides, diglycerides, cholesterol esters, free cholesterol, phospholipids and free fatty acids. In contrast, VLDL is a product of the liver and contains remodeled and repackaged triglycerides and cholesterol esters, as well as surface phospholipids, all of which, according to the inventors' hypothesis, may be influenced by the metabolic effects of HCV infection upon cellular lipid metabolism. Although for some of the identified various lipid biomarkers suitable for the assessment of the metabolic impact of HCV upon the liver cell their measurement in blood plasma may be liable to be confounded by the varying background of dietary lipids in the form of chylomicrons, some other identified markers, such as C54:6-C18:1 triglyceride (elevated 96-fold by infection) may still be useful if measured in unfractionated plasma, either in the fasted or postprandial state. Moreover, recent studies of the global fatty acid profile of human plasma have shown that the abundance of the various fatty acid species is controlled within narrow limits (Lamaziere, Wolf et al. 2012), suggesting that the effect of background dietary fluctuations on the lipid molecular profile biomarkers of hepatitis-C described here would not be so severe as to negate their use as biomarkers of the metabolic impact of HCV infection, although it is recognized that the straightforward global fatty acid profile of blood plasma may have limited usefulness for biomarker purposes (Flowers 2009). However, there may be at least two solutions to the confounding effect of dynamic variation in dietary plasma lipids.

Under fasting circumstances, VLDL is the predominant lipoprotein reservoir of triglycerides in the blood (Flowers 2009; Peter, Cegan et al. 2009). Thus, plasma triglyceride composition may be equivalent to VLDL triglyceride composition under fasting circumstances. Thus, at least with respect to the triglyceride molecular species profile characterized here as indicative of the effects of HCV infection on liver metabolism, one may expect, in the fasted state, that analysis of unfractionated plasma may be adequate for the biomarker purposes described here.

A second solution to the confounding problem of background dietary lipids may be a separation of VLDL from blood by an appropriate separation technique, such as density gradient ultracentrifugation or by a chromatographic method. Likewise (in the case of triglycerides) purification of triglycerides from blood plasma can be achieved by thin-layer chromatography or HPLC. Suitable methods for the isolation of VLDL and triglyceride fractions from human plasma are described in, for example, Peter et al. 2009.

With respect to measurement of the desaturation index (24:1/24:0) of glucosylceramide, it should be recognized that VLDL (being derived from liver) is also the appropriate blood lipoprotein to analyze, as for the other markers described above (e.g. particular triglyceride species). However, it is recognized that sphingomyelin is much more abundant among circulating sphingolipids than is glucosylceramide (Hammad, Pierce et al. 2010). In addition to measuring the desaturation of glucosylceramide in VLDL, it may be convenient or more sensitive to measure the 24:1/24:0 desaturation index of sphingomyelin, which the inventors have found to be affected by iminosugars in a similar way to glucosylceramide with respect to desaturation index.

### MATERIALS AND METHODS

Huh7.5 and Jc1 HCV Cell Culture:

Methods for cell culture of replication-competent HCV in human hepatoma cells were essentially as described (Pollock, Nichita et al. 2010). Huh7.5 cells (Apath, LLC) were grown in DMEM supplemented with 100 U/ml penicillin, 100 μg/ml streptomycin, 2 mM L-glutamine, 1×MEM, and 10% FBS. All incubations were at 37° C./5% CO₂. The effect of iminosugar treatment on cellular lipid profiles was determined for both uninfected and HCVcc-infected cells. In order to infect cells with HCV strain Jc1 (genotype 2a), Huh7.5 cells were incubated for 1 h in the presence of the virus at a multiplicity of infection (MOI)=0.02 using a viral stock of known titer. Cells were passaged for approximately 2 weeks to allow the infection to reach close to 100% of cells as determined by HCV core protein immunofluorescence, in order to avoid dilution of ‘infected’ lipidomic signatures by uninfected cells in partially infected cultures. Both HCV-infected and uninfected cells were then incubated in the presence or absence of iminosugars for 4 days, at which point they were harvested using trypsin/EDTA, washed 3 times in cold PBS, counted using trypan blue staining, and final cell pellets were resuspended in methanol:acetone (vol 1:1) prior to lipid profiling, A small volume of each sample was used for total protein estimation using the Bradford protein assay (Bio-Rad).

### Lipidomic Methods

“Total Lipid” Fatty Acid Profiling:

The procedure for “total lipid” FA measurements by GCMS has been described previously (Wolf 2008; Quinn, Rainteau et al. 2009) Briefly, pellets of cultured hepatoma Huh7.5 cells were extracted with chloroform using the method of Bligh & Dyer (Bligh and Dyer 1959). Briefly, chloroform lipid extracts of pelleted cultured hepatoma Huh7.5 cells was added with heptadecanoic acid as the internal standard. The solvent extract was dried in vacuo and the dry lipid film was transmethylated with methanol/H₂SO₄ (18N, 2% vol/vol) at 70° C. for 1 hour. An inert nitrogen/argon atmosphere, and addition of butylated hydroxytoluene (BHT) as antioxidant. Teflon-sealed disposable glass tubes were used to minimize peroxidation of polyunsaturated fatty acids. After cooling, water (1/2: vol/vol) was added and FA methyl esters (FAME) were extracted into hexane. The hexane extract was concentrated under a stream of nitrogen gas and transferred to an autosampler vial fitted with a 200 μl glass insert (Agilent 5975; 91940 Les Ulis, France). An aliquot of 1 μl was injected in the splitless mode of the GCMS apparatus (Agilent 5975; 91940 Les Ulis, France). Unsaturated FAME isomers (omega double-bond position at n3, n6, n7, n9) were separated on a polar bonded polyethylene-glycol capillary column (Omegawax; Sigma-Aldrich, L'Isle d'Abeau Chesnes 38297 Saint-Quentin Fallavier, France). Adducts (FAME+NH⁺₄) were assayed in chemical ionization mode with ammonia as the reagent gas (≈10⁻⁴ Torr, source temperature ≈100° C.). Quantification was performed by peak area integration after normalization relative to the internal standard (heptadecanoic acid) and calibration of the response coefficient with a Ponderal calibration mixture (Mix-37; Supelco-Sigma-Aldrich L'Isle d'Abeau Chesnes 38297 Saint-Quentin Fallavier, France).

Total Cholesterol (GCMS):

Total (esterified and non estified) cholesterol and sterol metabolites were derivatized to trimethylsilylether and profiled by GCMS as described in (Chevy, Illien et al. 2002; Chevy, Humbert et al. 2005). Briefly, the lipid chloroform extracts were added with d7-choroform (Avanti Polar Lipids, Lipid MS standards, Alabaster, Ala. 35007) and epicoprostanol (Sigma-Aldrich) as the internal standard. After fatty acid transmethylation as indicated above for the FAME preparation, the hexane extract was dried under a stream of nitrogen gas. Sterols were silylated for 60 min at 60° C. with 0.5 ml BSTFA (N,O-bis(trimethylsilyl)trifluoroacetamide) and TMCS 1% (trimethylchlorosilane) (Supelco Sigma-Aldrich 38297 Saint-Quentin-Fallavier Cedex). Excess reagent was evaporated and silylated sterols dissolved in hexane for GC injection. The separation of cholesterol and metabolites was performed between 200 and 250° C. on a medium polarity bonded diphenyl-dimethyl-polysiloxane capillary column (RTX50; Restek France Lisses France 9109). Detection and quantification relative to a Ponderal calibrator mixture was achieved by peak integration of characteristic ion-fragments in the positive mode (electron impact energy 70 eV).

LCMS2 Lipidomic Measurements:

The LCMS2 procedure has been detailed previously in methodological reviews (Ivanova, Milne et al. 2007; Myers, Ivanova et al. 2011). Briefly, the phospholipid chloroform extracts are prepared from pelleted Huh7.5 cells. A mixture of internal lipid standards was added to the extract (Avanti Polar Lipids, Lipid MAPS MS standards, Alabaster, Ala. 35007). The lipid classes were separated by HPLC (Agilent 1200 Series) on a polyvinyl-alcohol functionalized silica column (PVASil, YMC, ID 4 mm, length 250 mm, Interchim, Montluçon 03100, France). Less polar lipids (triglycerides, diglycerides, cholesterol esters, ceramides, glucosyl- and lactosylceramides) are eluted between 5 and 15 minutes by the solvent system hexane/isopropanol/water ammonium acetate 10 mM (40/58/2 vol/vol). Phospholipids were subsequently eluted by the solvent hexane/isopropanol/water ammonium acetate 10 mM (40/50/10 vol/vol) as a function of an increasing polarity between 15 and 60 minutes in the following order: phosphatidylethanolamine, lysophosphatidylethanolamine, phosphatidylserine, phosphatidylinositol, phosphatidylcholine, sphingomyelin, lysophosphatidylcholine. Eluted lipids were channeled into the electrospray interface of the spectrometer (Turbolon, Framingham, Mass. 01701, USA). The lipid ionization was run in positive mode for M+NH₄⁺ and M+H⁺ detection. The source was coupled to a triple quadrupole mass spectrometer (API3000, ABSciex, Toronto, Canada) run in the “collision induced dissociation” mode (or “precursor” mode) for monitoring the characteristic fragment ions of the successively eluted lipid classes. Precursor molecular species of the characteristic fragment ion were identified in a library prepared for cultured hepatoma cells with the software LIMSA (Haimi, Chaithanya et al. 2009). Molecular species of lipids being identified, a list of ion pairs (precursor/product ion) was prepared for quantification by multiple reaction monitoring (MRM). The corresponding MRM peaks are time-integrated. The lipid amounts were calculated relative to the appropriate lipid class standard assuming an even response coefficient of all molecular species in the class.

Statistical Methods:

Statistical procedures comparing lipid and fatty acid profiles were performed using the software XLStat® (version 2011. 2; Addinsoft, France). Parametric tests, multivariate analysis, correlation tests and regression procedures were applied as detailed (Golmard 2012).

### REFERENCES

Aizaki, H., K.-J. Lee, et al. (2004). Virology 324(2): 450-461.
Aizaki, H., K. Morikawa, et al. (2008). Journal of virology 82(12): 5715-5724.
Aizaki, H., K. Morikawa, et al. (2008). J Virol. 82(12): 5715-5724.
Amemiya, F., S. Maekawa, et al. (2008). The Journal of infectious diseases 197(3): 361-370.
Awad, T., K. Thorlund, et al. (2010). Hepatology 51(4): 1176-1184.
Barba, G., F. Harper, et al. (1997). Proc Natl Acad Sci USA. 94(4): 1200-1205.
Bernasconi, A. M., H. A. Garda, et al. (2000). Lipids 35(12): 1335-1344.
Berry, S. E. (2009). Nutrition research reviews 22(1): 3-17.
Bijl, N., M. Sokolovic, et al. (2009). Hepatology 50(5): 1431-1441.
Bligh, E. G. and W. J. Dyer (1959). Canadian journal of biochemistry and physiology 37(8): 911-917
Boot, R. G., M. Verhoek, et al. (2007). The Journal of biological chemistry 282(2): 1305-1312.
Branza-Nichita, N., D. Durantel, et al. (2001). Journal of virology 75(8): 3527-3536.
Burlone, M. E. and A. Budkowska (2009). The Journal of general virology 90(Pt 5): 1055-1070.
Butters, T. D., R. A. Dwek, et al. (2003). Advances in experimental medicine and biology 535: 219-226.
Butters, T. D., R. A. Dwek, et al. (2005). Glycobiology 15(10): 43R-52.
Castéra, L., J. Vergniol, et al. (2005). Gastroenterology 128(2): 343-350.
Chapel, C., C. Garcia, et al. (2007). J Gen Virol 88(4): 1133-1143.
Chapel, C., C. Garcia, et al. (2006). J Gen Virol 87(4): 861-871.
Chen, S. L. and T. R. Morgan (2006). International journal of medical sciences 3(2): 47.
Chevy, F., L. Humbert, et al. (2005). Prenatal diagnosis 25(11): 1000-1006.
Chevy, F., F. Illien, et al. (2002). Journal of lipid research 43(8): 1192-1200.
Cho, H. P., M. Nakamura, et al. (1999). The Journal of biological chemistry 274(52): 37335-37339.
Cho, H. P., M. T. Nakamura, et al. (1999). The Journal of biological chemistry 274(1): 471-477.
Clark, P. J. and A. J. Muir (2012). Hepatology 56(1): 5-8.
Clark, P. J., A. J. Thompson, et al. (2012). Hepatology 56(1): 49-56.
Clement, S., S. Pascarella, et al. (2009). Viruses 1(2): 126-143.
Diamond, D. L., A. J. Syder, et al. (2010). PLoS pathogens 6(1): e1000719.
Duffin, K., M. Obukowicz, et al. (2000). Analytical biochemistry 279(2): 179-188.
Eslam, M., M. A. Khattab, et al. (2011). Gut 60(8): 1139-1151.
Flowers, M. T. (2009). Clinical chemistry 55(12): 2071-2073.
Fox, T. E., M. C. Bewley, et al. (2011). Journal of lipid research 52(3): 509-517.
Fuller, M. (2010). Lipids in health and disease 9: 113.
Gangadharan, B., M. Bapat, et al. (2012). PloS one 7(6): e39603.
Golmard, J. L. (2012). Analyse Statistique des Donnees en Medecine & dans les Sciences de la Vie. Paris.
Haimi, P., K. Chaithanya, et al. (2009). Methods in molecular biology 580: 285-294.
Hammad, S. M., J. S. Pierce, et al. (2010). Journal of lipid research 51(10): 3074-3087.
Herker, E., C. Harris, et al. (2010). Nature medicine 16(11): 1295-1298.
Hollingsworth, R. C., P. Sillekens, et al. (1996). Journal of hepatology 25(3): 301-306
Huo, T. I., C. Y. Hsia, et al. (2007). Journal of surgical oncology 95(8): 645-651.
Ivanova, P. T., S. B. Milne, et al. (2007). Methods in enzymology 432: 21-57.
Kapadia, S. B., H. Barth, et al. (2007). J Virol. 81(1): 374-383.
Kapadia, S. B. and F. V. Chisari (2005). Proc Natl Acad Sci USA. 102(7): 2561-2566.
Keller S, Sanderson M P, Stoeck A, Altevogt P (2006). Immunol. Lett. 107 (2): 102-8
Lamaziere, A., C. Wolf, et al. (2012). Metabolites 2(1): 1-18.
Leu, G.-Z., T.-Y. Lin, et al. (2004). Biochem Biophys Res Commun 318(1): 275-280.
Lyn, R. K., D. C. Kennedy, et al. (2009). Virology 394(1): 130-142.
Marcellin, P. (1999). Journal of hepatology 31: 9-16.
Matto, M., C. M. Rice, et al. (2004). Journal of virology 78(21): 12047-12053.
McLauchlan, J. (2009). Biochimica et biophysica acta 1791(6): 552-559.
Merz, A., G. Long, et al. (2011). The Journal of biological chemistry 286(4): 3018-3032.
Miyoshi, H., K. Moriya, et al. (2011). Journal of hepatology 54(3): 432-438.
Moriishi, K. and Y. Matsuura (2012). Frontiers in microbiology 3: 54.
Muriana, F. J., C. M. Vazquez, et al. (1992). Journal of biochemistry 112(4): 562-567.
Myers, D. S., P. T. Ivanova, et al. (2011). Biochimica et biophysica acta 1811(11): 748-757.
Ntambi, J. M. (1999). Journal of lipid research 40(9): 1549-1558.
Ogawa, K., T. Hishiki, et al. (2009). Proceedings of the Japan Academy, Series B 85(7): 217-228.
Pawlotsky, J. M. (2011). Hepatology 53(5): 1742-1751.
Peter, A., A. Cegan, et al. (2009). Clinical chemistry 55(12): 2113-2120.
Platt, F. M., G. Reinkensmeier, et al. (1997). The Journal of biological chemistry 272(31): 19365-19372.
Pollock, S., N. B. Nichita, et al. (2010). Proceedings of the National Academy of Sciences 107(40): 17176-17181.
Quinn, P. J. (2010). Progress in lipid research 49(4): 390-406.
Quinn, P. J., D. Rainteau, et al. (2009). Methods in molecular biology 579: 127-159.
Rodgers, M. A., V. A. Villareal, et al. (2012). Journal of the American Chemical Society 134(16): 6896-6899.
Sagan, S. M., Y. Rouleau, et al. (2006). Biochemistry and cell biology=Biochimie et biologie cellulaire 84(1): 67-79.
Sainz, B., Jr., N. Barretto, et al. (2012). Nature medicine 18(2): 281-285.
Siguel, E. N., K. M. Chee, et al. (1987). Clinical chemistry 33(10): 1869-1873.
Syed, G. H., Y. Amako, et al. (2010). Trends in endocrinology and metabolism: TEM 21(1): 33-40.
Talukdar, I., W. Szeszel-Fedorowicz, et al. (2005). The Journal of biological chemistry 280(49): 40660-40667.
Wang, Y., D. Botolin, et al. (2006). Journal of lipid research 47(9): 2028-2041.
Waris, G., D. J. Felmlee, et al. (2007). J Virol. 81(15): 8122-8130.
Weng, L., Y. Hirata, et al. (2010). Journal of virology 84(22): 11761-11770.
Wolf, C., Quinn, P. J. (2008). Progress in lipid research 47: 15-36.
Ye, J. (2007). PLoS Pathog. 3(8): e108.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A method of assessing a Hepatitis C infection or a condition caused by or associated with said infection, said method comprising:
(a) obtaining a biological sample from a subject in need thereof;
(b) determining a level of at least one Hepatitis C lipidomic biomarker in said biological sample; and
(c) comparing said level of (b) with a control level of said Hepatitis C lipidomic biomarker to assess the Hepatitis C infection or the condition caused by or associated with said infection in the subject.

2. The method of claim 1, wherein said biological sample is a serum sample of the subject or a plasma sample of the subject.

3. The method of claim 1, wherein the subject is a human being.

4. The method of claim 1, wherein said determining said level comprises determining an abundance of Mead acid in said biological sample, wherein a lower value of the determined abundance compared to a control abundance value indicates that the subject has a Hepatitis C infection or a condition caused by or associated with said infection.

5. The method of claim 4, wherein a higher value of the determined abundance compared to a control abundance value indicates that the subject has Hepatocellular carcinoma.

6. The method of claim 4, wherein said determining the abundance of Mead acid comprising determining an abundance of Mead acid in a very low density protein fraction of the biological sample.

7. The method of claim 1, wherein said determining said level comprises determining an abundance of at least one of palmitoleic acid and oleic acid, wherein a lower value of the determined abundance compared to a control abundance value indicates that the subject has a Hepatitis C infection or a condition caused by or associated with said infection.

8. The method of claim 1, wherein said determining said level comprises determining a desaturation level of non-essential fatty acids in said biological sample, wherein a lower value of said desaturation level compared to a control desaturation level value indicates a Hepatitis C infection or a condition caused by or associated with said infection in said subject.

9. The method of claim 8, wherein said determining said level comprises determining a (16:1 ω-7+16:1ω-9)/16:0 ratio in the cells in said biological sample, wherein a lower value of the (16:1 ω-7+16:1ω-9)/16:0 ratio in the cells in said biological sample compared to a control (16:1 ω-7+16:1ω-9)/16:0 ratio value indicates a Hepatitis C infection or a condition caused by or associated with said infection in said subject.

10. The method of claim 1, wherein said determining said level comprises determining an elongation of non-essential fatty acids in said biological sample, wherein a higher value of said elongation in the biological sample compared to a control elongation value indicates a Hepatitis C infection or a condition caused by or associated with said infection in said subject.

11. The method of claim 1, wherein said determining comprises determining an abundance of at least one of polyunsaturated omega-6 and omega-3 fatty acids in said biological sample, wherein a higher value of said abundance in the biological sample compared to a control abundance value indicates a Hepatitis C infection or a condition caused by or associated with said infection in the subject.

12. The method of claim 11, wherein said determining comprises determining an abundance of at least one of arachidonic acid and docosahexaenoic acid, wherein a higher value of said abundance in the biological sample compared to a control abundance value indicates a Hepatitis C infection or a condition caused by or associated with said infection in the subject.

13. The method of claim 1, wherein said determining comprises determining a concentration of one or more fatty acids in a cholesterol ester profile of said biological sample.

14. The method of claim 13, wherein said one or more fatty acids comprise at least one polyunsaturated essential omega-3 and omega-6 fatty acid selected from a 20:4 fatty acid, a 20:5 fatty acid, a 22:6 fatty acid and a 22:5 fatty acid, wherein a higher value of the concentration of the polyunsaturated fatty acid in the cholesterol ester profile of the biological sample compared to a control cholesterol ester profile concentration of the polyunsaturated fatty acid indicates a Hepatitis C infection or a condition caused by or associated with said infection in the subject.

15. The method of claim 13, wherein said one or more fatty acids comprise at least one monounsaturated fatty acid selected from a 16:1 fatty acid or a 18:1 fatty acid, wherein an decrease in a value of the concentration of the monounsaturated fatty acid in the cholesterol ester profile of the cells of the biological sample compared to a control cholesterol ester profile concentration of the monounsaturated fatty acid indicates a Hepatitis C infection or a condition caused by or associated with said infection in the subject.

16. The method of claim 1, wherein said determining comprises determining a concentration of at least one triglyceride of said biological sample.

17. The method of claim 16, wherein said at least one triglyceride is selected from C54:5-C18:0 triglyceride; C54:6-C18:1 triglyceride; C56:5-C20:4 triglyceride and C56:7-C22:6 triglyceride, wherein a higher value of the concentration of said at least one triglyceride compared to a control value of said at least one triglyceride indicates a Hepatitis C infection or a condition caused by or associated with said infection in the subject.

18. The method of claim 16, wherein said determining comprises determining a concentration of C54:6-C18:1 triglyceride in said sample, wherein a higher value of said C54:6-C18:1 triglyceride compared to a control value of said C54:6-C18:1 triglyceride indicates a Hepatitis C infection or a condition caused by or associated with said infection in the subject.

19. The method of claim 18, wherein the biological sample is an unfractionated plasma sample of the subject.

20. The method of claim 16, wherein said obtaining is performed when the subject is in a fasted state.

21. The method of claim 1, wherein said determining comprises determining a concentration of at least one fatty acid among at least one of ester bonded phospholipids of the biological sample.

22. The method of claim 21, wherein said determining comprises determining a concentration of at least one fatty acid in a diester form of phosphatidylcholines of the biological sample, wherein the at least one fatty acid is selected from a PC 32:1 species, a PC 32:0 species, a PC 34:0 species, a PC 34:4 species and a PC 34:5 species, wherein a higher value of the concentration of at least one of the PC 32:0 species, the PC 34:0 species, the PC 34:4 species and the PC 34:5 species or a lower value of the concentration of the 32:1 species compared to a control value thereof indicates a Hepatitis C infection or a condition caused by or associated with said infection in the subject.

23. The method of claim 1, wherein said determining comprises determining a concentration of at least one fatty acid among at least one of lyso-phosphatidylcholines of the biological sample, wherein said fatty acid is selected from a 16:1 species, a 16:0 species, a 20:4 species and a 22:6 species, and wherein an higher value of the concentration of at least one of the 16:0 species, the 20:4 species and the 22:6 species or a lower value of the 16:1 species compared to a control value thereof indicates a Hepatitis C infection or a condition caused by or associated with said infection in the subject.

24. The method of claim 23, wherein said determining comprises determining a concentration of at least one fatty acid in a diester form of phosphatidylethanolamines of the cells of the biological sample, wherein the at least one fatty acid is selected from a) a Mead acid; b) at least one palmitoleic acids selected from 16:1 omega-7 and omega-9 acids; c) at least one of essential omega-3 or omega-6 fatty acids selected from 20:3 omega-3, 20:4 omega-6, 20:5 omega-3, 22:6 omega-3, 22:5 omega-3 and 22:4 omega-6 and wherein a higher value of the concentration of the Mead acid or the at least one essential omega-3 or omega-6 acid or a lower value of the concentration of the at least one palmitoleic acid compared to a control value thereof indicates a Hepatitis C infection or a condition caused by or associated with said infection in the subject.

25. The method of claim 24, wherein said determining comprises determining a concentration of at least one fatty acid in a diester form of phosphatidylserines of the biological sample, wherein the at least one fatty acid is selected from a 38:3 species and a 40:6 species and wherein a higher value of the concentration of the at least one of the 38:3 species and the 40:6 species compared to a control value thereof indicates a Hepatitis C infection or a condition caused by or associated with said infection in the subject.

26. The method of claim 21, wherein said determining comprises determining a concentration of at least one fatty acid in a diester form of phosphatidylionositol of the biological sample, wherein the at least one fatty acid is selected from a PI 38:3 species; a PI 36:4 species, a PI 38:4 species and a PI 38:5 species, wherein a higher value of the determined concentration of the PI 38:3 species or a lower value of the concentration of the at least one of the PI 36:4 species, the PI 38:4 species and the PI 38:5 species compared to a control value thereof indicates a Hepatitis C infection or a condition caused by or associated with said infection in the subject.

27. The method of claim 21, wherein said determining comprises determining a concentration of at least one fatty acid among at least one of ester bonded phosphadylcholines and ester bonded phosphatylethanolamine in a very low density lipoprotein fraction of the biological sample.

28. The method of claim 1, further comprising separating a very low density lipoprotein fraction of the biological sample and wherein said determining comprises determining the level of at least one Hepatitis C lipidomic biomarker in the very low density lipoprotein fraction of said biological sample.

29. The method of claim 26, wherein said separating is performed by density ultracentrifugation or by chromatography.

30. The method of claim 1, further comprising separating a triglyceride fraction of the biological sample and wherein said determining comprises determining the level of at least one Hepatitis C lipidomic biomarker in the trigliceride fraction of said biological sample.

31. The method of claim 1, wherein said determining comprises determining using at least one of gas chromatography with mass spectroscopy or liquid chromatography with mass spectroscopy.

32. The method of claim 1, which is a method of identifying, monitoring or assessing the severity of the Hepatitis C infection.

33. The method of claim 1, which is a method of assessing the progression or regression of the Hepatitis C infection.

34. The method of claim 1, which a method of determining an effect of an agent on the Hepatitis C infection, wherein the method further comprises administering said agent to the subject prior to said obtaining and wherein the control level is determined in a biological sample of the subject obtained prior to said administering.

35. The method of claim 1, which is a method of assessing a response to a treatment for the Hepatitis C infection, wherein the method further comprises administering the treatment to the subject prior to said obtaining and wherein the control level is determined in a biological sample of the subject obtained prior to said administering.

36. A method for assessing a response to a therapy, comprising:
(a) administering an agent to a subject in need thereof;
(b) then obtaining a biological sample from the subject;
(c) determining a desaturation index of at least one of glucosylceramide, lactosylceramide and sphingomyelin of the biological sample; and
(d) comparing a value of the desaturation index to a control desaturation index value to assess a response to said agent, wherein a higher value of the determined desaturation index compared to a control value indicates that the subject responds to the agent.

37. The method of claim 36, wherein said biological sample is a serum sample of the subject or a plasma sample of the subject.

38. The method of claim 36, wherein the subject is a human being.

39. The method of claim 36, further comprising separating a very low density lipoprotein fraction of the biological sample and wherein said determining comprises determining the level of the desaturation index in the very low density lipoprotein fraction of said biological sample.

40. The method of claim 39, wherein said separating is performed by density ultracentrifugation or by chromatography.

41. The method of claim 36, wherein said determining comprises determining a 24:1/24:0 ratio in the at least one of glucosylceramide and lactosylceramide in the lipoproteins of the biological sample.

42. The method of claim 36, wherein said determining comprising determining a desaturation index in both of glucosylceramide and lactosylceramide in the lipoproteins of the biological sample.

43. The method of claim 36, further comprising determining a concentration of glucosylceramide in the lipoproteins of the biological sample.

44. The method of claim 36, wherein the subject has at least one of Hepatitis C infection and genetic lysomal storage disorder.

45. The method of claim 44, wherein the subject has Gaucher disease or Niemann-Pick type-C disease.

46. The method of claim 44, wherein the agent comprises an iminosugar.

47. The method of claim 46, wherein said iminosugar is selected from N-substituted deoxynojrimycins and pharmaceutically acceptable salts thereof, N-substituted deoxygalactonojirimycins and pharmaceutically acceptable salts thereof and N-substituted Me-deoxygalactonojirimycins and pharmaceutically acceptable salts thereof.

48. The method of claim 47, wherein the iminosugar is selected from N-butyl deoxynojirimycin and a pharmaceutically acceptable salt thereof, methoxynonyl-deoxynojirimycin and a pharmaceutically acceptable salt thereof; N-(5-adamantane-1-yl-methoxypentyl)-DNJ and a pharmaceutically acceptable salt thereof; N-butyl deoxygalactonojirimycin and a pharmaceutically acceptable salt thereof; N-(7-oxa-nonyl)-1,5,6-trideoxy-1,5-imino-D-galactitol and a pharmaceutically acceptable salt thereof; and N—(N-{4′-azido-2′-nitrophenyl}-6-aminohexyl)deoxynojirimycin or a pharmaceutically acceptable salt thereof.

49. The method of claim 36, wherein the subject has a type II diabetes and the agent is an insulin sensitizing agent.

50. The method of claim 49, wherein the insulin sensitizing agent is selected from iminosugars, biguanides and thiazolidinediones.

51. A method of identifying of a Hepatitis C patient, who is unlikely to respond to a hepatitis C treatment comprising at least one of interferon and ribavirin, the method comprising:
(a) obtaining a biological sample from a subject having a Hepatitis C infection;
(b) determining a value of a desaturation index of at least one of glucosylceramide, lactosylceramide and sphingomyelin in lipoproteins of the biological sample; and
(c) comparing the determined value to a control desaturation index value, wherein if the determined value is higher than the control value of the desaturation, the subject is likely not to respond to a hepatitis C treatment comprising at least one of interferon and ribavirin.

52. The method of claim 51, wherein said biological sample is a serum sample of the subject or a plasma sample of the subject.

53. The method of claim 51, wherein the subject is a human being.

54. The method of claim 51, further comprising separating a very low density lipoprotein fraction of the biological sample and wherein said determining comprises determining the level of the desaturation index in the very low density lipoprotein fraction of said biological sample.

55. The method of claim 54, wherein said separating is performed by density ultracentrifugation or by chromatography.

56. The method of claim 54, wherein said determining comprises determining a 24:1/24:0 ratio in the at least one of glucosylceramide and lactosylceramide in the lipoproteins of the biological sample.

57. The method of claim 54, wherein said determining comprising determining a desaturation index in both of glucosylceramide and lactosylceramide in the lipoproteins of the biological sample.
